(19) United States
(12) Patent Application Publication
(10) Pub. No.: US 2023/0302045 A1
(43) Pub. Date: Sep. 28, 2023

(54) PHOSPHONATE-CONTAINING POLYMERS FOR VIRULENCE SUPPRESSION

(71) Applicant: 3M INNOVATIVE PROPERTIES COMPANY, St. Paul, MN (US)

(72) Inventors: Semra Colak Atan, St. Louis Park, MN (US); Hannah C. Cohen, St. Paul, MN (US); Max A. Kruziki, New Brighton, MN (US); Ranjani V. Parthasarathy, Woodbury, MN (US); Matthew T. Scholz, Woodbury, MN (US)

(21) Appl. No.: 18/004,940

(22) PCT Filed: Jun. 14, 2021
(86) PCT No.: PCT/IB2021/055218

§ 371 Date: Jan. 10, 2023

Related U.S. Application Data
(60) Provisional application No. 62/705,805, filed on Jul. 16, 2020.

(51) Int. Cl. A61K 31/80; A61P 31/04

(52) U.S. Cl. CPC A61K 31/80; A61P 31/04

(57) ABSTRACT

Medical compositions and methods of suppressing microbial virulence are provided. By suppressing virulence, administration and/or application of the medical compositions can be used to prevent, mitigate, or treat a microbial infection. More specifically, the medical compositions include a phosphonate-containing polymer. The phosphonate-containing polymers can suppress the expression of various virulence factors without destroying all microbes that may be present.

### BACKGROUND

Many known treatments of pathogens result in the destruction of all microbes that may be present, even beneficial microbes. Further, because of these treatment methods, there is growing concern about antibiotic resistance that will increase risks to patients, particularly to those undergoing surgical procedures. Newer approaches have been directed toward suppressing the virulence of the pathogen that causes the infection rather than destroying all microbes.

New methods are needed to prevent the expression of one or more virulence factors while preserving colonization of beneficial bacteria. That is, new methods are needed that do not destroy all the beneficial bacteria in the process of preventing the harm done by pathogens. The importance of phosphate-containing compositions for virulence suppression has been demonstrated in recent references such as in U.S. Pat. Publication 2019/0247423 (Alverdy et al.).

### SUMMARY

Medical compositions and methods of suppressing microbial virulence are provided. By suppressing virulence, administration and/or application of the medical compositions can be used to prevent, mitigate, or treat a microbial infection. More specifically, the medical compositions include a phosphonate-containing polymer. The phosphonate-containing polymers can suppress the expression of various virulence factors without destroying all microbes that may be present.

In a first aspect, a medical composition is provided that is suitable for preventing, mitigating, or treating a microbial infection. The medical composition includes a phosphonate-containing polymer having at least 0.8 mmoles phosphonate per gram of the phosphonate-containing polymer. The phosphonate-containing polymer is a polymerized reaction product of a monomer composition comprising a first monomer having (a) an ethylenically unsaturated group and (b) a phosphonate group of formula —P(═O)(OR¹)₂ or a salt thereof, wherein each R¹ is independently hydrogen, alkyl, aryl, aralkyl, or alkaryl.

In a second aspect, a method of suppressing microbial virulence is provided. The method includes administrating and/or applying a medical composition comprising a phosphonate-containing polymer having at least 0.8 mmoles phosphonate per gram of the phosphonate-containing polymer. The phosphonate-containing polymer is a polymerized reaction product of a monomer composition comprising a first monomer having (a) an ethylenically unsaturated group and (b) a phosphonate group of formula —P(═O)(OR¹)₂ or a salt thereof, wherein each R¹ is independently hydrogen, alkyl, aryl, aralkyl, or alkaryl.

As used herein, “alkyl” refers to a monovalent group that is a radical of an alkane and includes groups that are linear, branched, cyclic, bicyclic, or a combination thereof. Unless otherwise indicated, the alkyl groups typically contain from 1 to 20 carbon atoms. In some embodiments, the alkyl groups contain 1 to 10 carbon atoms, 2 to 10 carbon atoms, 1 to 6 carbon atoms, 2 to 6 carbon atoms, 1 to 4 carbon atoms, or 2 to 4 carbon atoms. Cyclic alkyl groups and branched alkyl groups have at least three carbon atoms. Examples of alkyl groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, adamantyl, norbornyl, and the like.

The term “alkylene” refers to a divalent group that is a radical of an alkane and includes groups that are linear, branched, cyclic, bicyclic, or a combination thereof. Unless otherwise indicated, the alkylene group typically has 1 to 20 carbon atoms. In some embodiments, the alkylene group has 1 to 10 carbon atoms, 2 to 10 carbon atoms, 1 to 6 carbon atoms, 2 to 6 carbon atoms, 1 to 4 carbon atoms, or 2 to 4 carbon atoms. Cyclic and branched alkylene groups have at least 3 carbon atoms. Suitable alkylene groups include, for example, methylene, ethylene, propylene, 1,4-butylene, 1,4-cyclohexylene, and 1,4-cyclohexyldimethylene.

The term “heteroalkylene” refers to an alkylene group that has at least one —CH₂— group replaced with a heteroatom such as sulfur, oxygen, or nitrogen. The heteroatom is typically in the form of an oxy group (—O—), thio group (—S—), or —NH— group. The heteroalkylene typically has at least one carbon atom (—CH₂— group) on either side of each heteroatom.

The term “aryl” refers to a monovalent group that is aromatic and, optionally but usually, carbocyclic. The aryl has at least one aromatic ring. Any additional rings can be unsaturated, partially saturated, saturated, or aromatic. Optionally, the aromatic ring can have one or more additional carbocyclic rings that are fused to or connected to the aromatic ring. Unless otherwise indicated, the aryl groups typically contain from 6 to 20 carbon atoms. In some embodiments, the aryl groups contain 6 to 18, 6 to 16, 6 to 12, or 6 to 10 carbon atoms. Examples of an aryl group include phenyl, naphthyl, biphenyl, phenanthryl, and anthracyl.

The term “aralkyl” refers to a monovalent group that is an alkyl group substituted with an aryl group (e.g., as in a benzyl group). The term “alkaryl” refers to a monovalent group that is an aryl substituted with an alkyl group (e.g., as in a tolyl group). Unless otherwise indicated, for both groups, the alkyl portion often has 1 to 10 carbon atoms, 1 to 6 carbon atoms, or 1 to 4 carbon atoms, and an aryl portion often has 6 to 20 carbon atoms, 6 to 18 carbon atoms, 6 to 16 carbon atoms, 6 to 12 carbon atoms, or 6 to 10 carbon atoms.

The term “ethylenically unsaturated” refers to a group having a double bond between the two carbon atoms at an end of the chemical structure. The ethylenically unsaturated group is typically either a vinyl group or a (meth)acryloyl group.

The term “(meth)acryloyl” refers to a monovalent group of formula CH₂═CR—(CO)— where R is hydrogen for an acryloyl group and methyl for a methacryloyl group and where —(CO)— refers to a carbonyl group.

As used herein, the term “phosphonate group” refers to a group of formula —P(═O)(OR¹)₂ that is bonded to a carbon atom and where R¹ is hydrogen, alkyl, aryl, aralkyl, or alkaryl. The term includes phosphonic acid groups (where at least one R¹ group is hydrogen), salts of phosphonic acid groups, and phosphonic acid ester groups (also known as phosphonate ester groups, where both R¹ groups are selected from alkyl, aryl, aralkyl, and aralkyl). The phosphonate group can be interchangeably written as —P(═O)(OR¹)₂ or —PO(OR¹)₂.

The term “monomeric unit” refers to a polymerized product of a monomer. For example, the monomeric unit associated with the monomer methyl acrylate (CH₂═CH—(CO)—O—CH₃) is

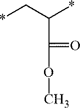 [SMILES: *CC(*)C(=O)OC]

where each asterisk (*) shows the location where the monomeric unit is attached to another monomeric unit or to a terminal group of a polymer.

The term “virulence” refers to a pathogen’s ability to infect or damage a host such as a mammal.

The term “virulence suppression” and “suppression of microbial virulence” or similar expressions refer to suppressing or inhibiting the synthesis and/or expression of one or more virulence factors.

The term “virulence factor” refers to molecules produced by microbes that enable them to infect a host such as a mammal. The virulence factors of bacteria can be small molecules, proteins, or biofilms (e.g., a slimy buildup of bacteria on a surface). The virulence factors are typically secreted by a microbe to promote colonization and/or adhesion to a host (e.g., resulting in biofilm formation), to evade the immune response of the host, or to obtain nutrients from the host.

The terms “comprise”, “contain”, “include”, and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of′ indicates that the listed elements are required or mandatory, and that no other elements may be present. By “consisting essentially of” is meant including any elements listed after the phrase and is limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether they materially affect the activity or action of the listed elements. Any of the elements or combinations of elements that are recited in this specification in open-ended language (e.g., comprise, include, contain, and derivatives thereof), are considered to additionally be recited in closed-ended language (e.g., consist and derivatives thereof) and in partially closed-ended language (e.g., consist essentially, and derivatives thereof).

In this application, terms such as “a”, “an”, and “the” are not intended to refer to only a singular entity but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise. The term “and/or” means one or both. For example, the expression A and/or B means A alone, B alone, or both A and B.

Also, the recitations of numerical ranges by endpoints include all numbers subsumed within that range as well as the endpoints (e.g., 1 to 5 includes 1, 1.5, 2, 2.75, 3, 3.80, 4, 5, etc.) and any sub-ranges (e.g., 1 to 5 includes 1 to 4, 1 to 3, 2 to 4, etc.).

### DETAILED DESCRIPTION

The importance of the amount of available phosphate in the proximity of bacteria has recently been demonstrated by various researchers. The virulence activity of certain bacteria such as Pseudomonas aeruginosa can increase if phosphate in their environment is scarce but can decrease if phosphate is abundant. For example, if a mammalian gut experiences a physiologic stress such as surgery, phosphate can become depleted. This depletion triggers colonized bacteria to express certain virulence factors. Hence, medical compositions that can provide a phosphate supply to the gut are critically needed. One approach to providing the phosphate supply is to administer a medical composition that can coat the gut and prevent the bacteria from turning virulent. Similarly, medical compositions that can supplement phosphate in a wound environment or surgical site would also be useful in keeping bacteria such as Pseudomonas aeruginosa from turning virulent.

Medical compositions and methods of suppressing microbial virulence are provided. Microbial virulence is suppressed by reducing or inhibiting the formation and/or expression of one or more virulence factors, which are the harmful products that can lead to microbial infections. That is, the medical compositions can prevent, mitigate, or treat microbial infections. More specifically, the medical compositions include a phosphonate-containing polymer. The medical composition typically does not prevent continued colonization of microbes such as those that are helpful to a mammal.

### Medical Composition

The medical composition includes a phosphonate-containing polymer having at least 0.8 mmoles phosphonate per gram of the phosphonate-containing polymer. Because of the increased hydrolytic stability of phosphonate groups compared to phosphate groups, the phosphonate-containing group is likely to remain in the desired biological environment and retain effectiveness for a greater time period. These phosphonate containing polymers also may be more resistant to enzymatic degradation than phosphate containing polymers. Other suitable optional components can be combined with the phosphonate-containing polymer to provide a medical composition that can be administered and/or applied for preventing, mitigating, or treating a microbial infection.

### Phosphonate-Containing Polymer

The phosphonate-containing polymer is a polymerized reaction product of a monomer composition comprising a first monomer having (a) an ethylenically unsaturated group and (b) a phosphonate group of formula —P(═O)(OR¹)₂ or a salt thereof, wherein each R¹ is independently hydrogen, alkyl, aryl, aralkyl, or alkaryl. The phosphonate-containing polymer can be a homopolymer that contains only first monomeric units or can be a copolymer that contains first monomeric units as well as other optional additional monomeric units.

Any monomer having both (a) an ethylenically unsaturated group and (b) a phosphonate group of formula —P(═O)(OR¹)₂ or a salt thereof can be used as the first monomer to prepare the phosphonate-containing polymer. The ethylenically unsaturated group is typically either a vinyl group or a (meth)acryloyl group. Both the selection of the ethylenically unsaturated group as well as the selection of the R¹ group can influence the miscibility of the phosphonate-containing polymer with water.

In many embodiments, the ethylenically unsaturated group is a (meth)acryloyl group and the first monomer is of Formula (I) or a salt thereof.

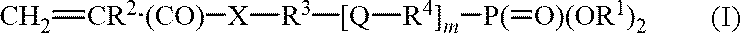

In Formula (I), each R¹ is independently hydrogen, aryl, aralkyl, or alkaryl. The group R² is hydrogen or methyl and the group X is oxy (—O—) or —NH—. Group R³ is either an alkylene or a heteroalkylene having one or more oxygen heteroatoms. Group R⁴ is an alkylene. Group Q is —(CO)—O—, —(CO)—NR⁵—, —NR⁵—(CO)—NR⁵—, or —O—(CO)—NR⁵— where each R⁵ is independently hydrogen or alkyl. The variable m is 0 or 1. The group —(CO)—X—R³—[—Q¹—R⁴—]ₘ—P(═O)(OR¹)₂ can be considered to be a pendant group of the first monomer.

The group —P(═O)(OR¹)₂ in Formula (I) can be a phosphonic acid group (where at least one R¹ is hydrogen), a salt of a phosphonic acid group, or a phosphonic acid ester (where each R¹ is an alkyl, aryl, aralkyl, or alkaryl). Any anionic group in the salt of the phosphonic acid group is charge balanced with a cationic group such as a cation of an alkaline metal or alkaline earth metal or a quaternary ammonium ion. The salt can be formed by treating the phosphonic acid group with a base. In many embodiments, each R¹ is independently hydrogen or an alkyl such as an alkyl with 1 to 10 carbon atoms, 1 to 6 carbon atoms, 1 to 4 carbon atoms, or 1 to 3 carbon atoms. Suitable aryl groups for R¹ often have 6 to 12 carbon atoms, 6 to 10 carbon atoms, or 6 to 8 carbon atoms. Suitable alkaryl and aralkyl R¹groups often have 7 to 12, 7 to 10, or 7 to 8 carbon atoms. Examples of aralkyl and alkaryl are benzyl and tolyl respectively.

In some embodiments of Formula (I), there are at least 6, at least 8, or at least 10 carbon atoms in the monomeric backbone between the terminal CH₂═CHR²— group and the phosphonate group (—P(═O)(OR¹)₂). This distance may permit better attachment of the phosphonate-containing polymer to the desired application and/or binding site.

When R² is hydrogen and X is oxy, the first monomer of Formula (I) is an acrylate. When R² is methyl and X is oxy, the monomer is a methacrylate. When R² is hydrogen and X is —NH—, the first monomer is an acrylamide and when R² is methyl and X is —NH—, the first monomer is a methacrylamide.

Group R³ is either an alkylene or a heteroalkylene having at least one oxygen heteroatom. Suitable alkylene groups often have 1 to 20 carbon atoms, 1 to 10 carbon atoms, 1 to 6 carbon atoms, or 1 to 4 carbon atoms. Suitable heteroalkylene groups often contain 2 to 20 carbon atoms, 2 to 10 carbon atoms, 2 to 6 carbon atoms, or 2 to 4 carbon atoms and 1 to 5 heteroatoms, 1 to 4 heteroatoms, of 1 to 3 heteroatoms.

Group Q is —(CO)—O—, —(CO)—NR⁵—, —NR⁵—(CO)—NR⁵—, or —NR⁵—(CO)—O— where R⁵ is hydrogen or alkyl. Suitable alkyl groups for R⁵ often have 1 to 10 carbon atoms, 1 to 6 carbon atoms, or 1 to 4 carbon atoms. In many embodiments, R⁵ is hydrogen or methyl. Group R⁵ is often hydrogen.

Group R⁴ is an alkylene. Suitable alkylene groups typically have 1 to 10 carbon atoms, 1 to 6 carbon atoms, 1 to 4 carbon atoms, or 1 to 3 carbon atoms.

When m is equal to 0, the first monomer of Formula (I) is of Formula (I-A).

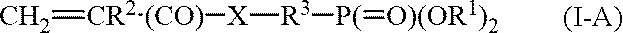

When m is equal to 1, the first monomer of Formula (I) is of Formula (I-B).

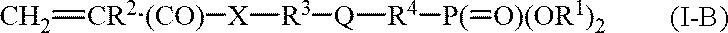

The groups R¹, R², X, R³, Q, and R⁴ are the same as for Formula (I).

The first monomer of Formula (I-A) can be a (meth)acrylate of Formula (I-A1) or a (meth)acrylamide of Formula (I-A2).

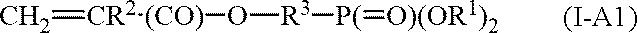

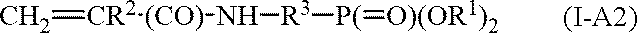

The groups R¹, R², and R³ are the same as described above for monomers of Formula (I).

The first monomers of Formula (I-A) can be prepared, for example, by reaction of (meth)acryloyl chloride with an equimolar amount of HX—R³—PO(OR¹)₂ to form a fist monomer of formula CH₂═CR²—(CO)—X—R³—PO(OR¹)₂. Groups X, R¹, R², and R³ are the same as described above. Suitable examples of compounds of formula HX—R³—PO(OR¹)₂ include hydroxyethylphosphonate dimethyl ester, aminomethyl phosphonic acid, aminoethyl phosphonic acid, and aminopropyl phosphonic acid.

The first monomers of Formula (I-A) having a phosphonic acid group also can be formed from the first monomer of formula CH₂═CR²—(CO)—X—R³—PO(OR^{1a})₂ having a phosphonic acid ester group. While R^{1a} can be an alkyl, aryl, aralkyl, or alkaryl, R^{1a} is often an alkyl. The phosphonic acid ester-containing monomer can be treated with bromotrimethylsilane to form an intermediate of formula CH₂═CR²—(CO)—X—R³—PO(OSi(CH₃)₃)₂ that is subsequently treated with an alcohol such as methanol to form the first monomer of formula CH₂═CR²—(CO)—X—R³—PO(OH)₂. Depending on the pH, the phosphonic acid group can become a phosphonate salt. For example, the phosphonic acid group can be treated with a base to be converted into a phosphonate salt.

The first monomer of Formula (I-B) can be a (meth)acrylate of Formula (I-B1) or a (meth)acrylamide of Formula (I-B2).

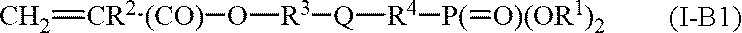

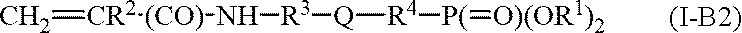

The groups R¹, R², Q, R³, and R⁴ are the same as for Formula (I).

The Q group in the first monomers of Formula (I-B) can be of formula —(CO)—O—, —(CO)—NR⁵—, —NR⁵—(CO)—NR⁵—, or —NR⁵—(CO)—O— where R⁵ is hydrogen or alkyl. Thus, the (meth)acrylate of Formula (I-B1) can be of Formula (I-B1-1), (I-B1-2), (I-B1-3), or (I-B1-4)

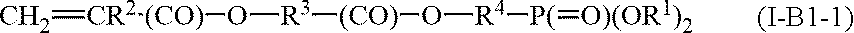

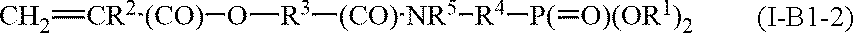

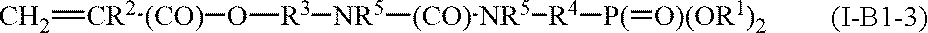

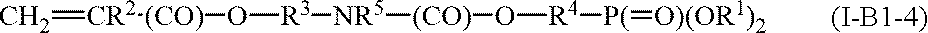

Likewise, the (meth)acrylamide of Formula (I-B2) can be of Formula (I-B2-1), (I-B2-2), (I-B2-3), or (I-B2-4).

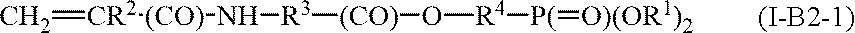

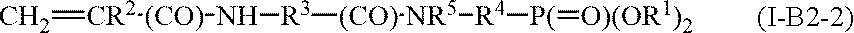

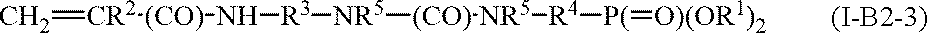

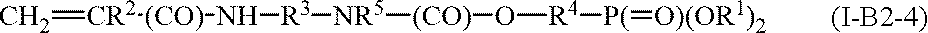

In some embodiments, the first monomer is of Formula (I-B1-3) or of Formula (I-B1-4). Such monomers can be prepared, for example, by reaction of an isocyanatoalkyl (meth)acrylate of formula CH₂═CR²—(CO)—O—R³—NCO with a compound of formula HX¹—R⁴—PO(OR¹)₂. Group X¹ is oxy or —NR⁵— where R⁵ is hydrogen or alkyl; group R⁵ is often hydrogen. That is, the monomers of Formulas (I-B1-3) and (I-B1-4) can be described by Formula (I-C).

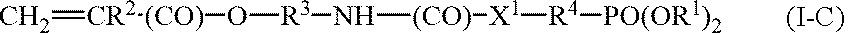

Groups R¹, R², and R³, R⁴ are the same as defined in Formula (I). The isocyanatoalkyl (meth)acrylate is often 2-isocyanatoethyl (meth)acrylate or 3-isocyanatopropyl (meth)acrylate. Examples of suitable compounds of formula HX¹—R⁴—PO(OR¹)₂ include hydroxyethylphosphonate dimethyl ester, hydroxyethylphosphonate diethyl ester, aminomethyl phosphonic acid, aminoethyl phosphonic acid, aminopropyl phosphonic acid. The phosphonic acid ester-containing monomers can be reacted with bromotrimethylsilane and then treated with an alcohol such as methanol to form the corresponding phosphonic acid-containing monomers.

In many embodiments of Formula (I-C), group R² is ethylene and the monomer of Formula (I-C) is of Formula (I-C-1) or (I-C-2).

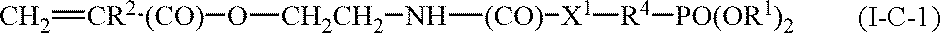

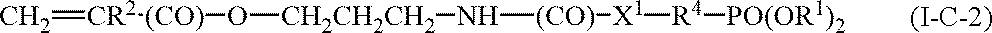

The group X¹ is usually oxy or —NH—.

In other embodiments, the first monomer is of Formula (I-B2-1) or (I-B2-2). Such monomers can be prepared by reaction of vinyl dimethyl azlactone (VDM) with a compound of formula HX¹—R⁴—PO(OR¹)₂. Groups X¹, R⁴, and R¹ are the same as described above. Examples of suitable compounds of formula HX¹—R⁴—PO(OR¹)₂ are the same as described above. The resulting monomers of Formula (I-D).

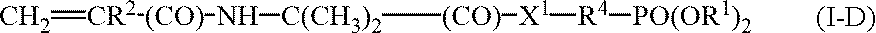

Groups R¹, R², and R⁴ are the same as defined in Formula (I). Group X¹ is oxy or —NR⁵— where R⁵ is hydrogen or alkyl; group R⁵ is usually hydrogen. The phosphonic acid ester-containing monomers can be reacted with bromotrimethylsilane and then treated with an alcohol such as methanol to form phosphonic acid-containing monomers.

The phosphonate-containing monomer can have a vinyl group rather than a (meth)acryloyl group. Such phosphonate-containing compounds are vinyl phosphonate are of Formula (II)

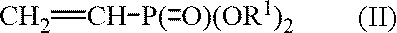

where R¹ is the same as described above for the monomers of Formula (I). In many embodiments of Formula (II), each R¹ is independently hydrogen or an alkyl such as methyl or ethyl.

The phosphonate-containing polymer can be a homopolymer where the monomeric units are all of Formulas (I) or (II). Alternatively, the phosphonate-containing polymer can be formed from a monomer composition that includes the first monomer of Formulas (I) or (II) plus one or more additional monomers that are different than the first monomer. That is, the monomer composition used to form the phosphonate-containing polymer can include up to 100 mole percent monomers of Formulas (I) or (II) based on the total moles of monomer in the monomer composition. The amount of the first monomer can be up to 99 mole percent, up to 98 mole percent, up to 97 mole percent, up to 95 mole percent, up to 90 mole percent, up to 85 mole percent, up to 80 mole percent, up to 75 mole percent, up to 70 mole percent, up to 65 mole percent, up to 60 mole percent, up to 55 mole percent, or up to 50 mole percent. The amount is typically greater than 25 mole percent, at least 30 mole percent, at least 35 mole percent, at least 40 mole percent, at least 45 mole percent, at least 50 mole percent, or greater than 50 mole percent, at least 55 mole percent, at least 60 mole percent, at least 65 mole percent, at least 70 mole percent, at least 75 mole percent, at least 80 mole percent, at least 85 mole percent, at least 90 mole percent, or at least 95 mole percent. The amount of the first monomer in the monomer composition is often in a range of 50 to 100 mole percent, greater than 50 to 100 mole percent, 55 to 100 mole percent, 60 to 100 mole percent, 70 to 100 mole percent, 75 to 100 mole percent, 80 to 100 mole percent, or 90 to 100 mole percent based on the total moles of monomer.

Additional monomers that are free of a phosphonate group can be included in the monomer composition. The additional monomers can be used to adjust the miscibility of the phosphonate-containing polymer in water or other solvent systems, which can include various organic solvents. If the addition monomers are (meth)acrylate-based monomers or (meth)acrylamide-based monomer, the resulting phosphonate-containing polymer will tend to be a random copolymer if the first monomer is of Formula (I) but a gradient or block-like copolymer if the first monomer is of Formula (II). That is, the rate of polymerization of the first monomer of Formula (II) may be slower than the rate of polymerization of the additional monomer.

In some embodiments, the monomer composition used to form the phosphonate-containing polymer includes a hydrophilic second monomer. Such phosphonate-containing polymers tend to be miscible in water, polar organic solvents, or a mixture thereof. In many embodiments, the term “hydrophilic” in reference to the second monomer means that the hydrophilic second monomer is soluble in distilled water in an amount of at least 10 weight percent, at least 15 weight percent, or at least 20 weight percent based on a total weight of the solution. As used herein, solubility of a monomer can be determined by adding a given amount of the monomer to water. If the monomer is completely dissolved in water (i.e., if the monomer is completely soluble in water), the resulting solution usually has at least 90 percent transmission at 550 nanometer (nm) through a one-centimeter (cm) path length.

Some hydrophilic second monomers contain an ethylenically unsaturated group plus a polar group such as an acidic group or salt thereof, a hydroxyl group, an ether (or polyether) group, or a nitrogen-containing group. Other hydrophilic second monomers contain an ethylenically unsaturated group plus a zwitterionic group. The ethylenically unsaturated group in the hydrophilic second monomer is typically a (meth)acryloyl group, particularly if the first monomer is of Formula (I).

Suitable hydrophilic second monomers with an acidic group include, for example, (meth)acrylic acid, β-caiboxyethyl (meth)acrylate, 2-(meth)acryloyloxyethyl phthalic acid, 2-(meth)acryloyloxy succinic acid, and combinations thereof. Various salts of these acidic groups may be present depending on the pH.

Exemplary hydrophilic second monomers with a hydroxyl group include, but are not limited to, hydroxyalkyl (meth)acrylates (e.g., 2-hydroxyethyl (meth)acrylate, 2-hydroxypropyl (meth)acrylate, 3-hydroxypropyl (meth)acrylate, and 4-hydroxybutyl (meth)acrylate), hydroxyalkyl (meth)acrylamides (e.g., 2-hydroxyethyl (meth)acrylamide and 3-hydroxypropyl (meth)acrylamide), ethoxylated hydroxyethyl (meth)acrylate (e.g., monomers commercially available from Sartomer (Exton, PA, USA) under the trade designation CD570, CD571, and CD572), aryloxy substituted hydroxyalkyl (meth)acrylates (e.g., 2-hydroxy-2-phenoxypropyl (meth)acrylate), 4-vinyl phenol, and hydroxy-propyl-carbamate acrylate.

Exemplary ether-containing (meth)acrylate monomers that can be used as a second hydrophilic second monomer include those selected from 2-ethoxyethoxyethyl (meth)acrylate, 2-methoxyethoxyethyl (meth)acrylate, di(ethylene glycol)-2-ethylhexyl-ether acrylate, ethylene glycol-methyl ether acrylate, and combinations thereof.

Exemplary hydrophilic second monomers with a primary amido group include (meth)acrylamide. Exemplary polar monomers with secondary amido groups include, but are not limited to, N-alkyl (meth)acrylamides or N-alkoxyalkyl (meth)acrylamides such as N-methyl (meth)acrylamide, N-ethyl (meth)acrylamide, N-isopropyl (meth)acrylamide, N-octyl (meth)acrylamide, N-(3-methoxypropyl)acrylamide, and N-(isobutoxymethyl)acrylamide. Exemplary polar monomers with a tertiary amido group include, but are not limited to, N-vinyl carbazole, N-vinyl caprolactam, N-vinyl-2-pyrrolidone, N-vinyl azlactone, 4-(meth)acryloylmorpholine, N-vinylimidazole, ureido (meth)acrylate, and N,N-dialkyl (meth)acrylamides such as N,N-dimethyl (meth)acrylamide, N,N-diethyl (meth)acrylamide, N,N-dipropyl (meth)acrylamide, and N,N-dibutyl (meth)acrylamide.

Hydrophilic second monomers with an amino group include various N,N-dialkylaminoalkyl (meth)acrylates and N,N-dialkylaminoalkyl (meth)acrylamides. Examples include, but are not limited to, N,N-dimethyl aminoethyl (meth)acrylate, N,N-dimethylaminoethyl (meth)acrylamide, N,N-dimethylaminopropyl (meth)acrylate, N,N-dimethylaminopropyl (meth)acrylamide, N,N-diethylaminoethyl (meth)acrylate, N,N-diethylaminoethyl (meth)acrylamide, N,N-diethylaminopropyl (meth)acrylate, and N,N-diethylaminopropyl (meth)acrylamide.

In other embodiments, the second monomer is a zwitterionic monomer. The zwitterionic second monomer is often of Formula (III).

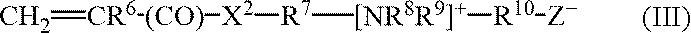

In Formula (III), R⁶ is hydrogen or methyl and X² is oxy or —NH—. Group R⁷ is an alkylene or a heteroalkylene having one or more oxygen heteroatoms. Groups R⁸ and R⁹ are each independently an alkyl, aryl, alkaryl or aralkyl, or R⁸ and R⁹ both combine with the nitrogen to which they are both attached to form a heterocyclic ring having 3 to 7 ring members. Group R¹⁰ is alkylene and Z⁻ is carboxylate or sulfonate.

When R⁶ is hydrogen and X² is oxy, the zwitterionic second monomer of Formula (III) is an acrylate. When R⁶ is methyl and X² is oxy, the monomer is a methacrylate. When R⁶ is hydrogen and X² is —NH—, the zwitterionic second monomer is an acrylamide and when R² is methyl and X² is —NH—, the monomer is a methacrylamide.

Group R⁷ is either an alkylene or a heteroalkylene having at least one oxygen heteroatom. Suitable alkylene groups often have 1 to 20 carbon atoms, 1 to 10 carbon atoms, 1 to 6 carbon atoms, or 1 to 4 carbon atoms. Suitable heteroalkylene groups often contain 2 to 20 carbon atoms, 2 to 10 carbon atoms, 2 to 6 carbon atoms, or 2 to 4 carbon atoms and 1 to 5 heteroatoms, 1 to 4 heteroatoms, of 1 to 3 heteroatoms.

In some embodiments, R⁸ and R⁹ are each independently an alkyl, aryl, aralkyl, or alkaryl. Suitable alkyl groups often have 1 to 10 carbon atoms, 1 to 6 carbon atoms, 1 to 4 carbon atoms, or 1 to 3 carbon atoms. Suitable aryl groups often have 6 to 12 carbon atoms, 6 to 10 carbon atoms, or 6 to 8 carbon atoms. Suitable alkaryl and aralkyl groups often have 7 to 12, 7 to 10, or 7 to 8 carbon atoms. An example aralkyl is benzyl. In other embodiments, R⁸ and R⁹ both combine with the nitrogen to which they are both attached to form a heterocyclic ring having 3 to 7 ring members. In addition to the nitrogen heteroatom, the heterocyclic ring can include another heteroatom selected from nitrogen, sulfur, or oxygen. In many embodiments R⁸ and R⁹ are each an alkyl group such as an alkyl group with 1 to 4 or 1 to 3 carbon atoms. The alkyl group is often methyl.

Group R¹⁰ is an alkylene. Suitable alkylene groups often have 1 to 10 carbon atoms, 1 to 6 carbon atoms, 1 to 4 carbon atoms, or 1 to 3 carbon atoms.

Group Z⁻ is typically either a carboxylate or a sulfonate.

The zwitterionic second monomer of Formula (III) can be a (meth)acrylate of Formula (III-A) or a (meth)acrylamide of Formula (III-B).

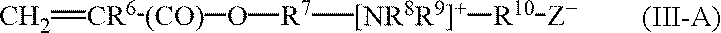

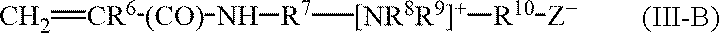

The groups R⁶, R⁷, R⁸, R⁹, R¹⁰, and Z⁻ are the same as described above for monomers of Formula (III).

In certain embodiments, the monomers of Formula (II-A) are selected from Formulas (III-A1) or (III-A2)

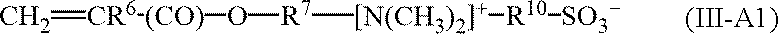

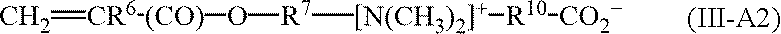

and the monomers of Formula (III-B) are selected from Formulas (III-B1) and (III-B2).

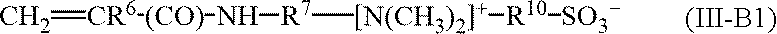

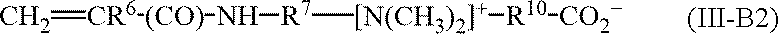

Zwitterionic second monomers of Formulas (III-A1) and (III-B1) where Z⁻ is sulfonate are commercially available. These include, for example, [2-(methacryloyloxy)ethyl]-dimethyl-(3-sulfopropyl)ammonium hydroxide and [3-(methacryloylamino)propyl]dimethyl(3-sulfopropyl)ammonium hydroxide. The zwitterionic second monomers of Formulas (III-A2) and (III-B2) where Z⁻ is carboxylate can be prepared, for example, by reacting of a compound of formula CH²═CR⁶—(CO)—X²—R⁷—NR⁸R⁹ with a compound of formula Br—R¹⁰—(CO)—O—R¹¹ where R¹¹ is an alkyl (e.g., an alkyl with 1 to 6 carbon atoms, 1 to 4 carbon atom, or 1 to 3 carbon atoms). The intermediate monomer can then be treated with sodium hydroxide to form a carboxylate Z⁻ group. An example zwitterionic monomer of Formula (III-B2) is 2-[dimethyl-[3-(prop-2-enoylamino)propyl]ammonio]acetate.

The monomer composition used to form the phosphonate-containing polymer usually includes less than 75 mole percent of the optional second monomer based on a total weight of monomers in the monomer composition. The amount, if present, can be up to 70 mole percent, up to 65 mole percent, up to 60 mole percent, up to 55 mole percent, up to 50 mole percent, less than 50 mole percent, up to 45 mole percent, up to 40 mole percent, up to 35 mole percent, up to 30 mole percent, up to 25 mole percent, up to 20 mole percent, up to 15 mole percent, or up to 10 mole percent.

In some embodiments, the monomer composition includes 100 mole percent of the first monomer. In other embodiments, the monomer composition includes greater than 25 mole percent of the first monomer and less than 75 mole percent of the second monomer, at least 30 mole percent of the first monomer and up to 70 mole percent of the second monomer, at least 40 mole percent of the first monomer and up to 60 mole percent of the second monomer, at least 50 mole percent of the first monomer and up to 50 mole percent of the second monomer, greater than 50 mole percent of the first monomer and less than 50 mole percent of the second monomer, at least 55 mole percent of the first monomer and up to 45 mole percent of the second monomer, at least 60 mole percent of the first monomer and up to 40 mole percent of the second monomer, at least 70 mole percent of the first monomer and up to 30 mole percent of the second monomer, at least 75 mole percent of the first monomer and up to 25 mole percent of the second monomer, at least 80 mole percent of the first monomer and up to 20 mole percent of the second monomer, or at least 90 mole percent of the first monomer and up to 10 mole percent of the second monomer.

The monomer composition may optionally further include a crosslinking monomer having two radically polymerizable groups. Crosslinking may be useful in lowering the solubility of the polymeric material after administration and/or application of the medical composition. Thus, the retention of the medical composition at the application and/or administration site may be enhanced by crosslinking the polymer. The crosslinking monomer is typically selected to be soluble and/or miscible with water or polar organic solvents.

For crosslinking of polymers prepared using (meth)acryloyl-containing monomers (such as those prepared using phosphonate-containing monomers of Formula (I)), the crosslinking monomer typically contains at least two (meth)acryloyl groups and can be a multifunctional (meth)acrylate, multifunctional (meth)acrylamide, or a mixture thereof. Example (meth)acryloyl-containing crosslinking monomers include, but are not limited to, ethyleneglycol di(meth)acrylate, 1,6-hexanediol di(meth)acrylate, poly(ethylene glycol) di(meth)acrylates, polybutadiene di(meth)acrylate, polyurethane di(meth)acrylates, propoxylated glycerin tri(meth)acrylate, methylenebisacrylamide, ethylenebisacrylamide, hexamethylenebisacrylamide, diacryloylpiperazine, and the like, and combinations thereof.

For crosslinking of polymers prepared using vinyl-containing monomers (such as those prepared using phosphonate-containing monomers of Formula (II)), the crosslinking monomer typically contains at least two allyl groups. Examples of allyl-containing crosslinking monomers include, but are not limited to, pentaerythritol allyl ether of formula C(CH₂OCH₂CH═CH₂)ₘ(CH₂OH)ₙ where m + n is equal to 4 and where n is at least 2), trimethylolpropane diallyl ether, glyoxal bis(diallyl acetal, allyl ether, and allyl sucrose.

The optional crosslinking monomer can be used in an amount ranging from 0 to 10 weight percent based on the total weight of monomers in the monomer composition. If used, the amount can be at least 0.01 weight percent, at least 0.05 weight percent, at least 0.1 weight percent, at least 0.2 weight percent, at least 0.5 weight percent, at least 1 weight percent, at least 2 weight percent, at least 3 weight percent, or at least 5 weight percent and up to 10 weight percent, up to 8 weight percent, up to 6 weight percent, up to 5 weight percent, up to 4 weight percent, up to 3 weight percent, up to 2 weight percent, or up to 1 weight percent.

In many embodiments, the monomer composition used to form the phosphonate-containing polymer contains greater than 25 to 100 weight percent first monomer, 0 to less than 75 weight percent second monomer, and 0 to 10 weight percent crosslinking monomer. In some examples, the monomer composition contains greater than 50 to 100 weight percent first monomer, 0 to less than 50 weight percent second monomer, and 0 to 10 weight percent crosslinking monomer. In another example, the monomer composition contains 60 to 100 weight percent first monomer, 0 to 40 weight percent second monomer, and 0 to 10 weight percent (or 0 to 5 weight percent) crosslinking monomer.

The phosphonate-containing polymer is typically prepared from a polymerizable composition that includes the monomer composition plus an initiator, which can be a photoinitiator or a thermal initiator. A polymerizable composition having a photoinitiator is often exposed to radiation in the ultraviolet and/or visible region of the electromagnetic spectrum for polymerization. A polymerizable composition having a thermal initiator is heated for polymerization at a temperature that is high enough for polymerization.

Thermal initiators for polymerization of the monomer composition can be water-soluble or water-insoluble (i.e., oil-soluble) depending on the polymerization method used. Suitable water-soluble initiators include, but are not limited to, persulfates such as potassium persulfate, ammonium persulfate, sodium persulfate, and mixtures thereof; an oxidation-reduction initiator such as the reaction product of a persulfate and a reducing agent such as a metabisulfite (e.g., sodium metabisulfite) or a bisulfate (e.g., sodium bisulfate); 4,4′-azobis(4-cyanopentanoic acid) and its soluble salts (e.g., sodium or potassium); or 4,4′-azobis(4-cyanovaleric acid) and its soluble salts (e.g., sodium or potassium). Suitable oil-soluble initiators include, but are not limited to, various azo compound such as those commercially available under the trade designation VAZO from E.I. DuPont de Nemours Co., (Wilmington, DE) including VAZO 67, which is 2,2′-azobis(2-methylbutane nitrile), VAZO 64, which is 2,2′-azobis(isobutyronitrile), and VAZO 52, which is (2,2′-azobis(2,4-dimethylpentanenitrile); and various peroxides such as benzoyl peroxide, cyclohexane peroxide, and lauroyl peroxide. Mixtures of various thermal initiators may be used if desired.

In many embodiments, a photoinitiator is used. Some exemplary photoinitiators are benzoin ethers (e.g., benzoin methyl ether or benzoin isopropyl ether) or substituted benzoin ethers (e.g., anisoin methyl ether). Other exemplary photoinitiators are substituted acetophenones such as 2,2-diethoxyacetophenone or 2,2-dimethoxy-2-phenylacetophenone (commercially available under the trade designation IRGACURE 651 from BASF Corp. (Florham Park, NJ) or under the trade designation ESACURE KB-1 from Sartomer (Exton, PA)). Still other exemplary photoinitiators are substituted alpha-ketols such as 2-methyl-2-hydroxypropiophenone, aromatic sulfonyl chlorides such as 2-naphthalenesulfonyl chloride, and photoactive oximes such as 1-phenyl-1,2-propanedione-2-(O-ethoxycarbonyl)oxime. Other suitable photoinitiators include, for example, 1-hydroxycyclohexyl phenyl ketone (IRGACURE 184), bis(2,4,6-trimethylbenzoyl)phenylphosphineoxide (IRGACURE 819), 1-[4-(2-hydroxyethoxy)phenyl]-2-hydroxy-2-methyl-1-propane-1-one (IRGACURE 2959), 2-benzyl-2-dimethylamino-1-(4-morpholinophenyl)butanone (IRGACURE 369), 2-methyl-1-[4-(methylthio)phenyl]-2-morpholinopropan-1-one (IRGACURE 907), and 2-hydroxy-2-methyl-1-phenyl propan-1-one (DAROCUR 1173). Additional photoinitiators include methyl 2,2-bis (isopropoxycarbothioylsulfanyl)acetate, polyethylene glycol 2,2-bis (isopropoxycarbothioylsulfanyl)acetate, and others disclosed in WO 2018/013330 (Griesgraber et al.). Mixtures of photoinitiators may be used if desired.

In certain embodiments, the polymerizable composition contains of 0.01 to 10 mole percent initiator based on the total moles of monomers in the monomer composition. The amount can be, for example, at least 0.01 mole percent, at least 0.05 mole percent, at least 0.1 mole percent, at least 0.5 mole percent, or at least 1 mole percent and up to 10 mole percent, up to 8 mole percent, up to 6 mole percent, up to 5 mole percent, up to 3 mole percent, or up to 1 mole percent.

The polymerizable composition may optionally further contain a chain transfer agent to control the molecular weight of the resultant polymer. Examples of useful chain transfer agents include, but are not limited to, carbon tetrabromide, alcohols, mercaptans such as isooctylthioglycolate, and mixtures thereof. If used, the polymerizable composition may include up to 0.5 weight of a chain transfer agent, based on a total weight of monomers in the monomer composition. For example, the polymerizable composition can contain 0.01 to 0.5 weight percent, 0.05 to 0.5 weight percent, or 0.05 to 0.2 weight percent chain transfer agent.

The phosphonate-containing polymer often has a theoretical (i.e., estimated) weight average molecular weight (Mw) of at least 2,000 Daltons, at least 5,000 Daltons, at least 8,000 Daltons, at least 10,000 Daltons, or at least 15,000 Daltons. In certain embodiments, the copolymer has a theoretical weight average molecular weight (Mw) of up to 20,000 Daltons, up to 50,000 Daltons, up to 100,000 Daltons, up to 150,000 Daltons, up to 200,000 Daltons, up to 500,000 Daltons, or even more. The theoretical weight average molecular weight may be determined by standard techniques including theoretical techniques (e.g., by evaluating a decreasing integration value for acrylate peaks corresponding to the starting monomers in NMR analysis). Alternatively, the weight average molecular weight can be determined on the final phosphate-containing polymer using methods known to those skilled in the art, include H-NMR characterization of the end groups or backbone hydrogen analysis, using aqueous size exclusion chromatography (SEC) with a multi-angle scattering light (MALS) detector, using an acid-base titration, or using aqueous gel permeation chromatograph.

The phosphonate-containing polymer contains at least 0.8 mmoles of phosphonate per gram of the phosphonate-containing polymer. The amount can be determined based on the theoretical content of phosphonate-containing monomers included in the monomer composition. Alternatively, the amount can be determined using an analytical method known to those skilled in the art such as, for example, P³¹-NMR or phosphorous elemental analysis (e.g., analysis using inductively coupled plasma spectroscopy or ion chromatography). The phosphonate-containing polymer often contains at least 0.8, at least 0.9, at least 1.0, at least 1.1, at least 1.2, at least 1.4, at least 1.5, at least 1.6, at least 1.8, or at least 2.0 mmoles of phosphonate per gram of the phosphonate-containing polymer. The upper amount can be dependent on the first monomer selected for use in preparing the phosphonate-containing polymer. If the first monomer is of Formula (II), the phosphonate-containing polymer can include up to 9.3 mmoles of phosphonate per gram of the phosphonate-containing polymer. Alternatively, if the first monomer is of Formula (I), the phosphonate-containing polymer can include up to 4.0 mmoles of phosphonate per gram of the phosphonate-containing polymer. The amount can be up to 9.3, up to 9.0, up to 8.5, up to 8.0, up to 7.5, up to 7.0, up to 6.5, up to 6.0, up to 5.5, up to 5.0, up to 4.5, up to 4.0, up to 3.8, up to 3.5, up to 3.4, up to 3.2, up to 3.0, up to 2.8, up to 2.6, up to 2.4, up to 2.2, or up to 2.0 mmoles of phosphonate per gram of the phosphonate-containing polymer.

One or a plurality of different phosphonate-containing polymers can be used in the medical compositions. Two or more different phosphonate-containing polymers can be blended together within the medical composition to suppress virulence of different types of pathogens and/or to suppress different virulence factors expressed by a single type of pathogen. For example, a first phosphonate-containing polymer that is particularly effective at suppressing the virulence of a first pathogen can be combined with a second phosphonate-containing polymer that is particularly effective at suppressing the virulence of a second pathogen. The plurality of different phosphonate-containing polymers can be blended together in any desired ratio.

The phosphonate-containing polymer, particularly those formed from a phosphonate-containing monomer of Formula (I), are usually water soluble. The solubility at room temperature (e.g., 20 to 25° C.) is typically at least 0.001 grams per mL and can be up to 2 grams per mL or even higher. The phosphonate-containing polymer is typically miscible with water, a polar organic solvent, or a mixture thereof.

The phosphonate-containing polymers are more hydrolytically stable than phosphate-containing polymers. The phosphonate-containing polymers are stable in an aqueous solution for at least one week, at least one month, at least two months, at least 3 months, at least 6 months, at least 12 months, at least 18 months, or at least 24 months when stored at room temperature (e.g., 20 to 25° C.).

The medical composition often contains 0.1 to 100 weight percent of the phosphonate-containing polymer based on a total weight of the medical composition. The amount can be at least 0.1 weight percent, at least 0.5 weight percent, at least 1 weight percent, at least 2 weight percent, at least 5 weight percent, at least 10 weight percent, at least 20 weight percent, at least 30 weight percent, at least 40 weight percent, or at least 50 weight percent and up to 100 weight percent, up to 90 weight percent, up to 80 weight percent, up to 70 weight percent, up to 60 weight percent, or up to 50 weight percent.

The phosphonate-containing copolymer can be purified, if desired, using any method that is suitable for purification of polymeric materials included in a medical composition. For example, the phosphate-containing copolymer can be purified by filtration.

### Optional Components

The medical composition includes the phosphonate-containing polymer and can optionally include other components that facilitate delivery of the medical composition for preventing, mitigating, or treating a microbial infection. The optional components are selected to be therapeutically acceptable, which means that the optional components do not interfere with the effectiveness of the phosphonate-containing polymer and are not toxic to the mammal being treated. The additional components are typically selected so that the medical composition does not substantially reduce non-pathogenic and/or normally helpful microbes that may be present. The log reduction of microbes is often less than 1.

The medical compositions can be delivered in any desired formulation such as a spray, lotion, ointment, gel, solution, emulsion, dispersion, foam, coating, paste, powder, tablet, capsule, adhesive (e.g., sealant), or the like. The formulation used is dependent on the location of the infection or potential infection and on the desired delivery method.

For some applications, it is desirable that the medical composition remain in a location where is it administered and/or applied. Such medical compositions are usually formulated to have a suitably high viscosity and to include a hydrophobic component that will enhance retention of the medical composition at the application location. These formulations can be, for example, an emulsion, ointment, gel, or lotion. Emulsions may be either oil-in-water or water-in-oil.

The medical compositions that include components such as, for example, water, organic solvents, hydrophobic components (e.g., petrolatum and oils), hydrophilic components (glycerin and various ether and/or polyether compounds), silicones, surfactants (i.e., anionic, cationic, nonionic, amphoteric, and ampholytic surfactants), carbohydrates, emulsifiers, water, organic solvents (e.g., alcohols and polyols), stabilizers (e.g., polymers), fillers (e.g., organic materials such as polymeric particles and inorganic materials including ceramic particles, silica particles, clay particles, and glass particles), emollients/ moisturizers, humectants, tonicity adjusting agents, chelating agents, anti-inflammatory agents, gelling agents, preservatives, pH adjusting agents, viscosity builders, time-release agents, dyes, fragrances or oils, and the like.

The medical compositions optionally can be sterilized by any suitable method that will not negatively impact its efficacy. For example, if desired, the medical composition can be treated with ethylene oxide.

### Method of Administering and/or Applying the Medical Composition

In another aspect, a method of suppressing microbial virulence is provided. The microbial virulence is typically suppressed by reducing or inhibiting the synthesis and/or expression of one or more virulence factors by the microbe. By suppressing the synthesis and/or expression of one or more virulence factors, a microbial infection can be prevented, mitigated, or treated.

The method includes administrating and/or applying a medical composition comprising an phosphonate-containing polymer having at least 0.8 mmoles phosphonate per gram of the phosphonate-containing copolymer, wherein the phosphonate-containing copolymer is a polymerized reaction product of a monomer composition comprising a first monomer having (a) an ethylenically unsaturated group and (b) a phosphonate group of formula —P(═O)(OR¹)₂ or a salt thereof, wherein each R¹ is independently hydrogen, alkyl, aryl, aralkyl, or alkaryl. Any of the medical compositions described above can be used.

Any suitable method of administering and/or applying the medical composition can be used. For example, the medical composition can be applied to skin, mucosa, tissue (both exterior and interior surfaces of tissue), a wound site, a surgical site, an implant (e.g., knee and hip replacement, pacemaker, heart valve, or stent), catheter, suture, or a bone.

The medical compositions can be administered and/or applied locally or systemically. For example, the medical compositions can be applied using a swab, cloth, sponge, nonwoven wipe, paper product such as a tissue or paper towel, or the like. Alternatively, the medical compositions can be delivered to the desired location using a tube, canula, or medical tool. When applied locally, the medical composition desirably remains where it was applied. That is, the medical composition persists at the location for enough time to suppress virulence of the pathogen. In other examples, the medical composition can be administered orally or intravenously. For some infections, such as those that are initiated in the gut, the medical composition can be administered by drinking a solution or by swallowing a tablet or capsule.

For treatment of wounds and surgical sites, application of the medical composition as a coating may be a desirable. Alternatively, the medical composition can be applied to a solid or porous support and then applied to the wound. Suitable supports include, for example, polymeric foams, polymeric films, and knitted or non-woven materials. The medical composition can be used for preventing and treating both acute and chronic wound infections and can be applied to any wound surface.

The medical composition can be administered and/or applied to reduce biofilm attachment on various surfaces. For example, the medical compositions can be applied to permanent or degradable implants or medical tools (e.g., endoscopes, catheters, and the like) prior to their insertion into a mammalian body. In other examples, the medical compositions can be applied to bedding, surgical tables, tubing used in medical procedures, and other reusable medical equipment that contacts a mammal. In yet other examples, the medical compositions can be a liquid composition that is used to control or prevent biofilm populations in oral applications, such as for treating gingivitis. In still other examples, the medical compositions can be used to control or prevent biofilm populations in the middle ear that have been found in chronic otitis media. In yet other examples, the medical compositions can be used to control or prevent biofilm populations in the nose, which can result in the prevention or treatment of various infections such as those in the lungs and blood. The medical compositions can often impact virulence factors either before or after biofilm formation.

The medical composition is suitable for preventing and treating urinary tract infections (e.g., administered in the form of a drink), ventilator associated pneumonia (e.g., administered in the form of a drink, tablet, or capsule), implant infections (e.g., administered by application as a coating on the implant), wounds (e.g., administered by application of a coating on the wound, whether chronic or acute), bloodstream infections (e.g., administered and/or applied to the blood-contacting tissue), mucosal tissue infections (e.g., administered in the nose), gastrointestinal tract (administered in the form of a coating, drink, tablet, or capsule), anastomotic tissue (e.g., administered as a coating on the surgical site to prevent anastomotic leaks), peritoneum (e.g., administered at the surgical site), sepsis, and the like. In some embodiments, where this is an existing microbial infection, the medical composition is applied over the area where the microbes are located.

The medical composition is usually administered in a therapeutically effective amount. This refers to the amount of the medical composition (or the amount of the phosphonate-containing polymer) that is needed to inhibit the synthesis and/or expression of one or more virulence factors by a microbe or that is enough to reduce, mitigate, or prevent a microbial infection.

Administering the medical composition suppresses at least one type of virulence factor. That is, the medical composition suppresses the formation and/or expression of various molecules that may be harmful to the mammal and/or suppresses the formation of biofilms on a foreign object such as an implant suture in the mammal. For example, the medical composition can suppress the formation and/or expression of pyocyanin, pyoverdine, collagenase (which is often measured by breakdown of gelatin as a surrogate of collagenase activity), and biofilms by bacteria.

In many embodiments of administering the medical composition, the virulence factor is reduced by at least 50 percent, at least 60 percent, at least 70 percent, at least 75 percent, at least 80 percent, at least 90 percent, at least 95 percent, at least 99 percent, at least 99.5 percent, or at least 99.9 percent when compared to the vehicle only control. The percentage can be based on weight, area, volume, or any other suitable measurable amount such as the intensity of a fluorescent or absorbance signal indicating virulence activity.

The medical composition may be suitable for treating any known microbe including, for example, bacteria, viruses, fungi such as Candida, and mycobacteria. In particular, administrating the medical composition can suppress virulence of at least one of gram negative Pseudomonas aeruginosa, gram positive Enterococcus faecalis, and gram positive Staphylococcus aureus.

Unlike some previously known methods of treating microbial infections, the medical composition does not substantially kill all microbes within the treatment area. Although some of the pathogens may be destroyed at the treatment site such as those associated with a biofilm, colonization of the protective microbes is not substantially reduced. Stated differently, the pathogens can be contained and controlled while the colonization resistance of the non-pathogenic microbes and/or the normally protective microbes can be preserved. As used in reference to reduction in the number of microbes that are present, the term “substantially” means that there is less than 1 log reduction of the microbes. In some embodiments, there may be in increase in the growth of protective microbes.

### EXAMPLES

### Materials

TABLE 1

Materials

| | |
|---|---|
| Description (Abbreviation) | Source |
| TY tryptone yeast medium (2X TY, as obtained, was diluted to 1X or to 10 weight percent TY) | Sigma-Aldrich Corporation, St. Louis, MO |
| DQ gelatin-fluorescein conjugate | Thermo Fisher Scientific, Waltham, MA |
| Chloroform | Avantor Performance Materials, Radnor, PA |
| TSA (Trypticase Soy Agar) Plates | Becton, Dickinson and Company, Franklin Lakes, NJ |
| Tryptic Soy Broth (TSB) | General Laboratory Products, Yorkville, IL |
| 2-Isocyanatoethyl methacrylate (IEM) | Showa Denko KK, Kanagawa, Japan |
| 4,4-Dimethyl-2-vinyl-4H-oxazol-5-one (vinyldimethylazlactone) (VDM, CAS# 29513-266) | SNPE, Inc, Princeton, NJ |
| 3-Aminopropyl phosphonic acid | Sinova, Beijing, China |
| Aminomethyl phosphonic acid | Sinova, Beijing, China |
| 3-Amino-1-propanesulfonic acid | Sigma-Aldrich Corporation |
| 3-Aminopropanoic acid | Sigma-Aldrich Corporation |
| [2-(Methacryloyloxy)ethyl]dimethyl-(3-sulfopropyl)ammonium hydroxide (SBMA) | Sigma-Aldrich Corporation |
| 4,4-azobis-4-cyanovaleric acid | Pfaltz & Bauer, Waterbury, CT |
| Poly(vinylphosphonic acid) | Sigma-Aldrich Corporation (CAS Number 27754-99-0 corresponding to product number 661740) |

Unless otherwise noted, all other reagents were obtained from the Sigma-Aldrich Corporation (St. Louis, MO).

Unless otherwise noted, all aqueous compositions were prepared with 18 MΩ water from a water purification system (available under the trade designation “Milli-Q” from EMD Millipore, Billerica, MA).

### Media Preparations

Phosphate deficient medium (PDM) was prepared as a solution containing 0.5 mM MgSO₄, 0.1 mM 4-(2-Hydroxyethyl)piperazine-1-ethanesulfonic acid (HEPES) pH 7.0, 7 mM (NH₄)₂SO₄, 20 mM disodium succinate, 0.1 mM KH₂PO₄, and a trace ion mixture that contained 0.1% of 2.45 mM CaCl₂, 13.91 mM ZnCl₂, 4.69 mM H₃BO₄, 0.67 mM CoCl₂, and 1.78 mM FeSO₄ dissolved in water.

Defined citrate media (DCM) was prepared as a solution containing 4.0 g/L sodium citrate, 1 g/L (NH₄)₂SO₄, and 0.2 g/L MgSO₄ × 7 H₂O, in 0.1 mM potassium phosphate buffer.

### Preparatory Examples

### Monomer A

[methyl-2-(prop-2-enoylamino)propanoyl]amino]methylphosphonic acid (VDM- NC₁-PA)

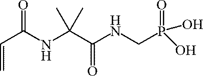 [SMILES: C=CC(=O)NC(C)(C)C(=O)NCP(=O)(O)O]

Aminomethylphosphonic acid (22.2 g, 0.2 mol) was added to a 500-mL round bottom flask. An aqueous solution of sodium hydroxide (1.0 N, 400 mL) was added to the flask and the resulting mixture was stirred until the solids dissolved. The flask was then placed in an ice-water bath and stirred for 30 minutes. VDM (27.8 g, 0.2 mol) was added dropwise via syringe and the reaction was stirred for 30 minutes with the flask continuously maintained in the ice-water bath. The cooling bath was then removed, and the reaction was allowed to warm to room temperature over a period of 1 hour. A small amount of precipitate was removed by filtration. The pH of the filtrate was adjusted to about 7 by the addition of a few drops of a concentrated hydrochloric acid solution. ¹H-NMR of an aliquot of the filtrate confirmed the formation of [[2-methyl-2-(prop-2-enoylamino)propanoyl]amino]methylphosphonic acid. ¹H-NMR (D₂O, 500 MHz) δ 1.38 (s, 6H), 3.07 (d, 2H), 5.62 (d, 1H), 6.02-6.20 (m, 2H).

### Monomer B

3-[Methyl-2-(prop-2-enoylamino)propanoyl]amino]propylphosphonic acid (VDM- NC₃-PA)

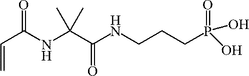 [SMILES: C=CC(=O)NC(C)(C)C(=O)NCCCP(=O)(O)O]

3-Aminopropylphosphonic acid (20.9 g, 0.15 mol) was added to a 500-mL round bottom flask. An aqueous solution of sodium hydroxide (1.0 N, 300.0 mL) was added to the flask and the resulting mixture was stirred until the solids dissolved. The flask was then placed in an ice-water bath and stirred for 30 minutes. VDM (20.9 g, 0.15 mol) was added dropwise via syringe and the reaction was stirred for 30 minutes with the flask continuously maintained in the ice-water bath. The cooling bath was then removed, and the reaction was allowed to warm to room temperature over a period of 1 hour. A small amount of precipitate was removed by filtration. The pH of the filtrate was adjusted to about 7 by the addition of a few drops of a concentrated hydrochloric acid solution. ¹H-NMR of an aliquot of the filtrate confirmed the formation of 3-[[2-methyl-2-(prop-2-enoylamino)propanoyl]amino]propylphosphonic acid. ¹H-NMR (D₂O, 500 MHz) δ 1.19-1.26 (m, 2H), 1.34 (s, 6H), 1.48-1.57 (m, 2H), 3.06 (t, 2H), 5.62 (d, 1H), 6.0-6.2 (m, 2H).

### Monomer C

(2-Methylprop-2-enoyloxy)ethylcarbamoylamino]methylphosphonic acid (IEM- NC₁-PA)

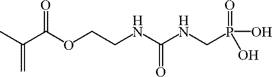 [SMILES: C=C(C)C(=O)OCCNC(=O)NCP(=O)(O)O]

Aminomethylphosphonic acid (22.2 g, 0.2 mol) was added to a 500-mL round bottom flask. An aqueous solution of sodium hydroxide (1.0 N, 400.0 mL) was added to the flask and the resulting mixture was stirred until the solids dissolved. The flask was then placed in an ice-water bath and stirred for 30 minutes. IEM (31.0 g, 0.2 mol) was added dropwise via syringe and the reaction was stirred for 30 minutes with the flask continuously maintained in the ice-water bath. The cooling bath was then removed, and the reaction was allowed to warm to room temperature over a period of 1 hour. A small amount of precipitate was removed by filtration. The pH of the filtrate was adjusted to about 7 by the addition of a few drops of a concentrated hydrochloric acid solution. ¹H-NMR of an aliquot of the filtrate confirmed the formation of 3-[2-(2-methylprop-2-enoyloxy)ethylcarbamoylamino]propylphosphonic acid. ¹H-NMR (D₂O, 500 MHz) δ 1.79 (s, 3H), 3.01-3.07 (m, 2H), 3.29-3.36 (m, 2H), 4.09 (t, 2H), 5.58 (s, 1H), 6.01 (s, 1H).

### Monomer D

3-(2-Methylprop-2-enoyloxy)ethylcarbamoylamino]propylphosphonic acid (IEM- NC₃-PA)

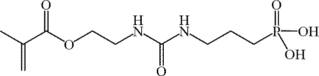 [SMILES: C=C(C)C(=O)OCCNC(=O)NCCCP(=O)(O)O]

3-Aminopropylphosphonic acid (20.9 g, 0.15 mol) was added to a 500-mL round bottom flask. An aqueous solution of sodium hydroxide (1.0 N, 300.0 mL) was added to the flask and the resulting mixture was stirred until the solids dissolved. The flask was then placed in an ice-water bath and stirred for 30 minutes. IEM (23.3 g, 0.15 mol) was added dropwise via syringe and the reaction was stirred for 30 minutes with the flask continuously maintained in the ice-water bath. The cooling bath was then removed, and the reaction was allowed to warm to room temperature over a period of 1 hour. A small amount of precipitate was removed by filtration. The pH of the filtrate was adjusted to about 7 by the addition of a few drops of a concentrated hydrochloric acid solution. ¹H-NMR of an aliquot of the filtrate confirmed the formation of 3-[2-(2-methylprop-2-enoyloxy)ethylcarbamoylamino]propylphosphonic acid. ¹H-NMR (D₂O, 500 MHz) δ 1.20-1.27 (m, 2H), 1-47-1.55 (m, 2H), 1.79 (s, 3H), 2.97 (t, 2H), 3.31 (t, 2H), 4.09 (t, 2H), 5.58 (s 1H), 5.99 (s, 1H).

### Monomer E

3-(2-methylprop-2-enoyloxy)ethylcarbamoylamino]propane-1-sulfonic acid (IEM-NC₃-S)

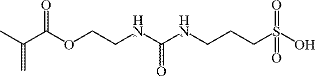 [SMILES: C=C(C)C(=O)OCCNC(=O)NCCCS(=O)(=O)O]

3-Aminopropanesulfonic acid (3.5 g, 0.025 mol) was added to a 100-mL round bottom flask. An aqueous solution of sodium hydroxide (1.0 N, 25.0 mL) was added to the flask and the resulting mixture was stirred until the solids dissolved. The flask was then placed in an ice-water bath and stirred for 30 minutes. IEM (3.9 g, 0.025 mol) was added dropwise via syringe and the reaction was stirred for 30 minutes with the flask continuously maintained in the ice-water bath. The cooling bath was then removed, and the reaction was allowed to warm to room temperature over a period of 1 hour. A small amount of precipitate was removed by filtration. The pH of the filtrate was adjusted to about 7 by the addition of a few drops of a concentrated hydrochloric acid solution. ¹H-NMR of an aliquot of the filtrate confirmed the formation of 3-[2-(2-methylprop-2-enoyloxy)ethylcarbamoylamino]propane-1-sulfonic acid. ¹H NMR (D₂O, 500 MHz) δ 1.74 - 1.81 (m, 5H) 2.77 - 2.81 (m, 2H) 3.07 - 3.13 (m, 2H) 3.31 - 3.35 (m, 2H) 4.11 (t, 2H) 5.59 - 5.62 (m, 1H) 6.02 (s, 1H).

### Monomer F

3-(2-methylprop-2-enoyloxy)ethylcarbamoylamino]propanoic acid (IEM-NC₂-C)

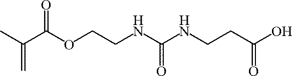 [SMILES: C=C(C)C(=O)OCCNC(=O)NCCC(=O)O]

3-Aminopropanoic acid (18.8 g, 0.2 mol) was added to a 500-mL round bottom flask. An aqueous solution of sodium hydroxide (1.0 N, 200.0 mL) was added to the flask and the resulting mixture was stirred until the solids dissolved. The flask was then placed in an ice-water bath and stirred for 30 minutes. IEM (31.0 g, 0.2 mol) was added dropwise via syringe and the reaction was stirred for 30 minutes with the flask continuously maintained in the ice-water bath. The cooling bath was then removed, and the reaction was allowed to warm to room temperature over a period of 1 hour. A small amount of precipitate was removed by filtration. The pH of the filtrate was adjusted to about 7 by the addition of a few drops of a concentrated hydrochloric acid solution. ¹H-NMR of an aliquot of the filtrate confirmed the formation of 3-[2-(2-methylprop-2-enoyloxy)ethylcarbamoylamino]propanoic acid. ¹H NMR (D₂O, 500 MHz) δ 1.78 (s, 3H) 2.22 (t, 2H)3.17(t,2H) 3.31 (t, 2H) 4.08 (t, 2H) 5.57 - 5.61 (m, 1H) 6.00 (s, 1H).

### Polymer Preparation

The extent of polymerization was determined by ¹H-NMR analysis. All polymers of the examples showed 90-99% consumption of (meth)acrylate/(meth)acrylamide monomers. All polymers were dialyzed using a Biotech Cellulose Ester (CE) Dialysis Membrane (obtained from Spectrum Laboratories, Rancho Dominguez, CA) with Molecular Weight Cut Off (MWCO):3.5-5 kD (wet in 0.05% sodium azide) for the high molecular weight samples (>10 kD) and MWCO:500-1,000 D for the low molecular weight samples (<6,000 D). The samples were dialyzed using Milli-Q grade water for 48 hours.

### Calculation of Phosphonate Concentration (Mmol Per Gram of Polymer)

The concentration of phosphonate (mmol) per gram of polymer was calculated based on the theoretical molecular weight of the polymer and the number of phosphonate repeat units according to the following equation:

$$\begin{matrix}\frac{m m o l P h o s p h o n a t e}{g o f P o l y m e r} = \\ \frac{\# o f R e p e a t U n i t s o f P h o s p h o n a t e i n t h e P o l y m e r \times 1000}{T h e o r e t i c a l M o l e c u l a r W e i g h t o f t h e P o l y m e r}\end{matrix}$$

where the # of repeat units were determined according to the following equation:

$$\begin{matrix}\# o f R e p e a t U n i t s o f P h o s p h o n a t e i n t h e P o l y m e r = \\ \frac{\left[m o l s o f P h o s p h o n a t e M o n o m e r\right]}{2 ∗ \left[m o l s o f I n i t i a t o r\right]}\end{matrix}$$

and the theoretical molecular weight of the polymer has been calculated according to the following equation:

$$\begin{matrix}T h o r e t i c a l M o l e c u l a r W e i g h t o f t h e P o l y m e r \\ = \sum {\left(\# o f R e p e a t U n i t s o f M o n o m e r\right)}^{i} ∗ \\ {\left(M o l e c u l a r W e i g h t o f M o n o m e r\right)}^{i}\end{matrix}$$

where the superscript i is equal to the number of different monomers in the polymer.

### Example 1: 5K Poly(VDM-NC₃-PA)

A polymerization solution was prepared by mixing a solution of Monomer B (9.68 g of 14.7% solids solution, 4.42 mmol) with the initiator 4,4-azobis-4-cyanovaleric acid (0.036 g, 0.13 mmol) in a 30-mL clear glass vial. The reaction mixture was purged with a stream of nitrogen for 15 minutes. The vial was then closed with a screw cap and placed on a hot plate at 85° C. The polymerization was conducted at the set temperature for 12 hours. ¹H-NMR of an aliquot showed a monomer conversion of ≥ 95%. The estimated molecular weight of the polymer was approximately 5,475 Da. The concentration of phosphonate (mmol) per gram of polymer was calculated to be 3.1 mmol/g polymer.

### Example 2: 5K Poly(VDM-NC₁-PA)

A polymerization solution was prepared by mixing a solution of Monomer A (9.35 g of 13.9% solids solution, 4.42 mmol) with the initiator 4,4-azobis-4-cyanovaleric acid (0.036 g, 0.13 mmol) in a 30-mL clear glass vial. The procedure described in Example 1 was followed to provide the final polymer. ¹H-NMR of an aliquot showed a monomer conversion of ≥ 95%. The estimated molecular weight of the polymer was approximately 4,998 Da. The concentration of phosphonate (mmol) per gram of polymer was calculated to be 3.4 mmol/g polymer.

### Example 3: 5K Poly(IEM-NC₁-PA)

A polymerization solution was prepared by mixing a solution of Monomer C (9.29 g of 14.8% solids solution, 4.42 mmol) with the initiator 4,4-azobis-4-cyanovaleric acid (0.036 g, 0.13 mmol) in a 30-mL clear glass vial. The procedure described in Example 1 was followed to provide the final polymer. ¹H-NMR of an aliquot showed a monomer conversion of ≥ 95%. The estimated molecular weight of the polymer was approximately 5,270 Da. The concentration of phosphonate (mmol) per gram of polymer was calculated to be 3.2 mmol/g polymer.

### Example 4: 5K Poly(IEM-NC₃-PA)

A polymerization solution was prepared by mixing a solution of Monomer D (10.13 g of 14.8% solids solution, 4.42 mmol) with the initiator 4,4-azobis-4-cyanovaleric acid (0.036 g, 0.13 mmol) in a 30-mL clear glass vial. The procedure described in Example 1 was followed to provide the final polymer. ¹H-NMR of an aliquot showed a monomer conversion of ≥ 95%. The estimated molecular weight of the polymer was approximately 5,746 Da. The concentration of phosphonate (mmol) per gram of polymer was calculated to be 3.0 mmol/g polymer.

### Example 5: 10K Poly(VDM-NC₃-PA)

A polymerization solution was prepared by mixing a solution of Monomer B (19.37 g of 14.7% solids solution, 8.84 mmol) with the initiator 4,4-azobis-4-cyanovaleric acid (0.036 g, 0.13 mmol) in a 30-mL clear glass vial. The procedure described in Example 1 was followed to provide the final polymer. ¹H-NMR of an aliquot showed a monomer conversion of ≥ 95%. The estimated molecular weight of the polymer was approximately 10,950 Da. The concentration of phosphonate (mmol) per gram of polymer was calculated to be 3.1 mmol/g polymer.

### Example 6: 10K Poly(VDM-NC₁-PA)

A polymerization solution was prepared by mixing a solution of Monomer A (18.7 g of 13.9% solids solution, 8.84 mmol) with the initiator 4,4-azobis-4-cyanovaleric acid (0.036 g, 0.13 mmol) in a 30-mL clear glass vial. The procedure described in Example 1 was followed to provide the final polymer. ¹H-NMR of an aliquot showed a monomer conversion of ≥ 95%. The estimated molecular weight of the polymer was approximately 9,996 Da. The concentration of phosphonate (mmol) per gram of polymer was calculated to be 3.4 mmol/g polymer.

### Example 7: 10K Poly(IEM-NC₁-PA)

A polymerization solution was prepared by mixing a solution of Monomer C (18.58 g of 14.8% solids solution, 8.84 mmol) with the initiator 4,4-azobis-4-cyanovaleric acid (0.036 g, 0.13 mmol) in a 30-mL clear glass vial. The procedure described in Example 1 was followed to provide the final polymer. ¹H-NMR of an aliquot showed a monomer conversion of ≥ 95%. The estimated molecular weight of the polymer was approximately 10,540 Da. The concentration of phosphonate (mmol) per gram of polymer was calculated to be 3.2 mmol/g polymer.

### Example 8: 10K Poly(IEM-NC₃-PA)

A polymerization solution was prepared by mixing a solution of Monomer D (20.26 g of 14.8% solids solution, 8.84 mmol) with the initiator 4,4-azobis-4-cyanovaleric acid (0.036 g, 0.13 mmol) in a 30-mL clear glass vial. The procedure described in Example 1 was followed to provide the final polymer. ¹H-NMR of an aliquot showed a monomer conversion of ≥ 95%. The estimated molecular weight of the polymer was approximately 11,492 Da. The concentration of phosphonate (mmol) per gram of polymer was calculated to be 3.0 mmol/g polymer.

### Example 9: 15K Poly(VDM-NC₃-PA)

A polymerization solution was prepared by mixing a solution of Monomer B (28.49 g of 14.7% solids solution, 13.0 mmol) with the initiator 4,4-azobis-4-cyanovaleric acid (0.036 g, 0.13 mmol) in a 30-mL clear glass vial. The procedure described in Example 1 was followed to provide the final polymer. ¹H-NMR of an aliquot showed a monomer conversion of ≥ 95%. The estimated molecular weight of the polymer was approximately 16,103 Da. The concentration of phosphonate (mmol) per gram of polymer was calculated to be 3.1 mmol/g polymer.

### Example 10: 15K Poly(VDM-NC₁-PA)

A polymerization solution was prepared by mixing a solution of Monomer A (27.50 g of 13.9% solids solution, 13.0 mmol) with the initiator 4,4-azobis-4-cyanovaleric acid (0.036 g, 0.13 mmol) in a 30-mL clear glass vial. The procedure described in Example 1 was followed to provide the final polymer. ¹H-NMR of an aliquot showed a monomer conversion of ≥ 95%. The estimated molecular weight of the polymer was approximately 14,700 Da. The concentration of phosphonate (mmol) per gram of polymer was calculated to be 3.4 mmol/g polymer.

### Example 11: 15K Poly(IEM-NC₁-PA)

A polymerization solution was prepared by mixing a solution of Monomer C (27.32 g of 14.8% solids solution, 13.0 mmol) with the initiator 4,4-azobis-4-cyanovaleric acid (0.036 g, 0.13 mmol) in a 30-mL clear glass vial. The procedure described in Example 1 was followed to provide the final polymer. ¹H-NMR of an aliquot showed a monomer conversion of ≥ 95%. The estimated molecular weight of the polymer was approximately 15,500 Da. The concentration of phosphonate (mmol) per gram of polymer was calculated to be 3.2 mmol/g polymer.

### Example 12: 15K Poly(IEM-NC₃-PA)

A polymerization solution was prepared by mixing a solution of Monomer D (29.8 g of 14.8% solids solution, 13.0 mmol) with the initiator 4,4-azobis-4-cyanovaleric acid (0.036 g, 0.13 mmol) in a 30-mL clear glass vial. The procedure described in Example 1 was followed to provide the final polymer. ¹H-NMR of an aliquot showed a monomer conversion of ≥ 95%. The estimated molecular weight of the polymer was approximately 16,900 Da. The concentration of phosphonate (mmol) per gram of polymer was calculated to be 3.0 mmol/g polymer.

### Example 13: 15K Poly(IEM-NC₃-PA-co-SBMA) (50:50)

A polymerization solution was prepared by mixing a solution of Monomer D (14.1 g of 15.6% solids solution, 6.5 mmol), SBMA (1.82 g, 6.5 mmol), and the initiator 4,4-azobis-4-cyanovaleric acid (0.036 g, 0.13 mmol) in a 30-mL clear glass vial. The solution was further diluted with 13 mL of 0.5 M NaCl solution. The procedure described in Example 1 was followed to provide the final polymer. ¹H-NMR of an aliquot showed a monomer conversion of ≥ 95%. The estimated molecular weight of the copolymer was approximately 15,000 Da. The concentration of phosphonate (mmol) per gram of copolymer was calculated to be 1.6 mmol/g polymer.

### Example 14: 15K Poly(IEM-NC₃-PA-co-SBMA) (75:25)

A polymerization solution was prepared by mixing a solution of Monomer D (10.6 g of 15.6% solids solution, 4.9 mmol), SBMA (0.45 g, 1.6 mmol), and the initiator 4,4-azobis-4-cyanovaleric acid (0.018 g, 0.07 mmol) in a 30-mL clear glass vial. The solution was further diluted with 15 mL of 0.5 M NaCl solution. The procedure described in Example 1 was followed to provide the final polymer. ¹H-NMR of an aliquot showed a monomer conversion of ≥ 95%. The estimated molecular weight of the copolymer was approximately 16,200 Da. The concentration of phosphonate (mmol) per gram of copolymer was calculated to be 2.3 mmol/g polymer.

### Example 15: Poly(vinyl)phosphonic Acid

Poly(vinylphosphonic acid) was purchased from Sigma-Aldrich Corporation. The concentration of phosphonate (mmol) per gram of polymer was calculated to be 9.3 mmol/g polymer. As determined by GPC, the number average molecular weight was 3370 Daltons, the weight average molecular weight was 10,300 Daltons.

### Comparative Example A: 15K Poly(IEM-NC₂-C)

A polymerization solution was prepared by mixing a solution Monomer F (20.9 g of 21.1% solids solution, 13.0 mmol) with the initiator 4,4-azobis-4-cyanovaleric acid (0.036 g, 0.13 mmol) in a 30-mL clear glass vial. The procedure described in Example 1 was followed to provide the final polymer. ¹H-NMR of an aliquot showed a monomer conversion of ≥ 95%. The estimated molecular weight of the polymer was approximately 16,900 Da.

### Comparative Example B: 15K Poly(IEM-NC₃-S)

A polymerization solution was prepared by mixing a solution of Monomer E (15.6 g of 24.5% solids solution, 13.0 mmol) with the initiator 4,4-azobis-4-cyanovaleric acid (0.036 g, 0.13 mmol) in a 30-mL clear glass vial. The procedure described in Example 1 was followed to provide the final polymer. ¹H-NMR of an aliquot showed a monomer conversion of ≥ 95%. The estimated molecular weight of the polymer was approximately 14,700 Da.

### Comparative Example C: 15K Poly(SBMA)

A polymerization solution was prepared by mixing a solution of SBMA (3.63 g, 13.0 mmol) with the initiator 4,4-azobis-4-cyanovaleric acid (0.036 g, 0.13 mmol) in a 30-mL clear glass vial. The solution was further diluted with 25 mL of 0.5 M NaCl solution. The procedure described in Example 1 was followed to provide the final polymer. ¹H-NMR of an aliquot showed a monomer conversion of ≥ 95%. The estimated molecular weight of the polymer was approximately 14,000 Da.

### Example 16: Pyocyanin Assay

Individual growth media solutions for the assay were prepared by adding 0.5 weight percent (wt.%) of a single polymer selected from Examples 1-2, 5-9, 12-14 and Comparative Examples A-C to PDM and then adjusting the pH of each solution to about pH 6.0 (using 1 M NaOH or 1 M HCl). Solutions were sterile filtered when possible using a 0.2 micrometer filter.

An MPAO1-P2 Pseudomonas aeruginosa colony from a TSA plate was grown overnight in TSB media with shaking at 37° C. The overnight culture was diluted 1:50 in fresh TSB and grown with shaking at 37° C. until the OD600 (absorbance at 600 nm) reached 0.5. The culture was split into equal volumes in multiple tubes, centrifuged at 10,000 × g for 5 minutes, and the supernatants were removed. The resulting bacteria pellet in each tube was resuspended by adding one of the growth media solutions (about 2 mL) to the tube. A control sample was also prepared, by resuspending the bacteria pellet in a tube with PDM (2 mL) that did not contain added phosphonate-containing polymer. The sample tubes were cultured overnight with shaking at 37° C. Following the culture step, the tubes were observed by visual examination to determine if the solution in the tube had a blue color. The presence of a blue colored solution at the completion of the assay indicated secretion of pyocyanin by Pseudomonas aeruginosa in a test sample. The results are presented in Table 2.

TABLE 2

Pyocyanin Production from MPAO1-P2 P. aeruginosa

| | |
|---|---|
| Polymer (0.5 wt.%) Added to Growth Media (PDM) | Blue Color Observed |
| None (control) | Yes |
| Example 1 | No |
| Example 2 | No |
| Example 5 | No |
| Example 6 | No |
| Example 7 | No |
| Example 8 | No |
| Example 9 | No |
| Example 12 | No |
| Example 13 | No |
| Example 14 | No |
| Comparative Example A | Yes |
| Comparative Example B | Yes |
| Comparative Example C | Yes |

### Example 17: Pyoverdine Assay

An MPAO1-P1 Pseudomonas aeruginosa colony from a TSA plate was grown overnight in 1X TY media (5 mL) with shaking at 37° C.

Individual growth media solutions for the assay were freshly prepared by adding 0.5 wt.% of a single polymer selected from Examples 1-4 to 10% TY media and then adjusting the pH of each solution to about pH 6.0 (using 1 M NaOH or 1 M HCl). The solutions were sterile filtered when possible using a 0.2 micrometer filter.

Each growth media solution (200 microliters) was added to a separate well of a 96-well black, clear-bottom plate. Samples were prepared in triplicate (n=3). MPAO1-P1 bacteria were centrifuged and resuspended in 5 mL of 10% TY media and 3 microliters of resuspended bacteria was added to each well. Background control wells were also prepared that did not have bacteria added to the wells. Pyoverdine production (fluorescent intensity at 360 nm excitation/ 460 nm emission) and bacteria growth (OD600; i.e., absorbance at 600 nm) were measured kinetically using a plate reader (Synergy HTX Plate Reader, Biotek Instruments, Winooski, VT) with shaking at 37° C. The background values were subtracted from the respective fluorescence and absorbance measurements. The pyoverdine fluorescence values (RFU, relative fluorescence units) were then normalized for bacteria growth by dividing the RFU value by the OD600 measurement. In Tables 3-4, data is shown at the 24 hour time point. The test samples with added phosphonate-containing polymer had significantly less pyoverdine production than the control samples that had no added polymer (Table 3). Compared to the control samples, the test samples with added phosphonate-containing polymer did not substantially reduce bacterial growth (Table 4).

TABLE 3

Pyoverdine Production/Bacteria Growth at 24 hours (MPAO1-P1 P. aeruginosa)

| Polymer (0.5 wt.%) Added to Growth Media (10% TY Media) | Pyoverdine Production (RFU/OD600) (24 hours) |
|---|---|
| | Mean ± Standard Deviation (n = 3) |
| None (control) | 352 ± 34 |
| Example 1 | 47 ± 3 |
| Example 2 | 139 ± 8 |
| Example 3 | 95 ± 26 |
| Example 4 | 24 ± 6 |

TABLE 4

Bacteria Growth (OD600) at 24 hours (MPAO1-P1 P. aeruginosa)

| Polymer (0.5 wt.%) Added to Growth Media (10% TY Media) | Bacteria Growth (OD600) (24 hours) |
|---|---|
| | Mean ± Standard Deviation (n = 3) |
| None (control) | 0.177 ± 0.007 |
| Example 1 | 0.153 ± 0.006 |
| Example 2 | 0.153 ± 0.001 |
| Example 3 | 0.38 ± 0.10 |
| Example 4 | 0.25 ± 0.02 |

### Example 18: Pyoverdine Assay

The same procedure as described in Example 17 was followed with the exception that test samples were prepared containing a lower concentration (0.1 wt%) of a single phosphonate-containing polymer selected from Examples 1-12 in defined citrate media (DCM), instead of 10% TY growth media. The test samples with added phosphonate-containing polymer had significantly less pyoverdine production than the control samples that had no added polymer (Table 5). Compared to the control samples, the test samples with added phosphonate-containing polymer did not substantially reduce bacterial growth (Table 6).

TABLE 5

Pyoverdine Production/Bacteria Growth at 24 hours (MPAO1-P1 P. aeruginosa)

| Polymer (0.1 wt.%) Added to Growth Media (DCM) | Pyoverdine Production (RFU/OD600) (24 hours) |
|---|---|
| | Mean ± Standard Deviation (n = 3) |
| None (control) | 287413 ± 5622 |
| Example 1 | 3991 ± 798 |
| Example 2 | 150711 ± 16239 |
| Example 3 | 240051 ± 19365 |
| Example 4 | 1866 ± 198 |
| Example 5 | 3599 ± 617 |
| Example 6 | 185491 ± 40268 |
| Example 7 | 206696 ± 51365 |
| Example 8 | 17959 ± 1104 |
| Example 9 | 4886 ± 1045 |
| Example 10 | 166238 ± 4236 |
| Example 11 | 173374 ± 18013 |
| Example 12 | 25789 ± 1243 |

TABLE 6

Bacteria Growth (OD600) at 24 hours (MPAO1-P1 P. aeruginosa)

| Polymer (0.1 wt.%) Added to Growth Media (DCM) | Bacteria Growth (OD600) (24 hours) |
|---|---|
| | Mean ± Standard Deviation (n = 3) |
| None (control) | 0.118 ± 0.019 |
| Example 1 | 0.092 ± 0.016 |
| Example 2 | 0.144 ± 0.015 |
| Example 3 | 0.187 ± 0.022 |
| Example 4 | 0.147 ± 0.002 |
| Example 5 | 0.084 ± 0.012 |
| Example 6 | 0.134 ± 0.017 |
| Example 7 | 0.196 ± 0.045 |
| Example 8 | 0.248 ± 0.003 |
| Example 9 | 0.067 ± 0.004 |
| Example 10 | 0.119 ± 0.008 |
| Example 11 | 0.189 ± 0.025 |
| Example 12 | 0.342 ± 0.021 |

### Example 19: Pyoverdine Assay

The same procedure as described in Example 17 was followed with the exception that test samples, prepared using a single polymer selected from Examples 1-4, were evaluated with MPAO1-P2 bacteria, instead of MPAO1-P1 bacteria. The test samples with added phosphonate-containing polymer had significantly less pyoverdine production than the control samples that had no added polymer (Table 7). Compared to the control samples, the test samples with added phosphonate-containing polymer did not substantially reduce bacterial growth (Table 8).

TABLE 7

Pyoverdine Production/Bacteria Growth at 24 hours (MPAO1-P2 P. aeruginosa)

| Polymer (0.5 wt.%) Added to Growth Media (10% TY Media) | Pyoverdine Production (RFU/OD600) (24 hours) |
|---|---|
| | Mean ± Standard Deviation (n = 3) |
| None (control) | 193 ± 5 |
| Example 1 | 26 ± 9 |
| Example 2 | 88 ± 15 |
| Example 3 | 87 ± 34 |
| Example 4 | 4.0 ± 0.8 |

TABLE 8

Bacteria Growth (OD600) at 24 hours (MPAO1-P2 P. aeruginosa)

| Polymer (0.5 wt.%) Added to Growth Media (10% TY Media) | Bacteria Growth (OD600) (24 hours) |
|---|---|
| | Mean ± Standard Deviation (n = 3) |
| None (control) | 0.152 ± 0.005 |
| Example 1 | 0.293 ± 0.087 |
| Example 2 | 0.183 ± 0.008 |
| Example 3 | 0.269 ± 0.076 |
| Example 4 | 0.663 ± 0.026 |

### Example 20: Pyoverdine Assay

The same procedure as described in Example 19 was followed with the exception that test samples, prepared using a single polymer selected from Examples 12-14 or Comparative Examples A-C, were evaluated at the 15 hour time point, instead of the 24 hour time point (n=5). The test samples with added phosphonate-containing polymer had significantly less pyoverdine production than the control samples that had no added polymer (Table 9). Compared to the control samples, the test samples with added phosphonate-containing polymer did not reduce bacterial growth (Table 10).

TABLE 9

Pyoverdine Production/Bacteria Growth at 15 hours (MPAO1-P2 P. aeruginosa)

| Polymer (0.5 wt.%) Added to Growth Media (10% TY Media) | Pyoverdine Production (RFU/OD600) (15 hours) |
|---|---|
| | Mean ± Standard Deviation (n = 5) |
| None (control) | 877 ± 85 |
| Example 12 | 25 ± 5 |
| Example 13 | 49 ± 16 |
| Example 14 | 34 ± 11 |
| Comparative Example A | 335 ± 62 |
| Comparative Example B | 598 ± 116 |
| Comparative Example C | 526 ± 52 |

TABLE 10

Bacteria Growth (OD600) at 15 hours (MPAO1-P2 P. aeruginosa)

| Polymer (0.5 wt.%) Added to Growth Media (10% TY Media) | Bacteria Growth (OD600) (15 hours) |
|---|---|
| | Mean ± Standard Deviation (n = 5) |
| None (control) | 0.103 ± 0.009 |
| Example 12 | 0.297 ± 0.039 |
| Example 13 | 0.236 ± 0.053 |
| Example 14 | 0.258 ± 0.056 |
| Comparative Example A | 0.116 ± 0.036 |
| Comparative Example B | 0.110 ± 0.022 |
| Comparative Example C | 0.184 ± 0.017 |

### Example 21: Collagenase Assay Using Gelatin Breakdown as a Surrogate

Breakdown of tissue proteins has been observed in many bacterial infections and is attributed to bacterial collagenase activity. Gelatin breakdown was used as a surrogate for collagen breakdown in the assay. A fluorescently labeled gelatin (DQ gelatin-fluorescein conjugate) was used to monitor gelatin breakdown with the fluorescence signal increasing with increased gelatin degradation.

An MPAO1-P2 Pseudomonas aeruginosa colony and an Enterococcus faecalis (V583) colony were each obtained from a corresponding TSA plate and separately grown overnight in 1X TY media (5 mL) with shaking at 37° C. Next, each culture was centrifuged at 3000 × g for 5 minutes and the supernatant was removed. The bacteria were washed two times with water.

For assays using MPAO1-P2 Pseudomonas aeruginosa, individual growth media solutions for the assay were freshly prepared by adding 0.5 wt.% of a single polymer selected from Examples 1-4, 0.05 wt.% of Example 15, or combinations of polymers selected from Examples 5 and 7 to 10% TY media and then adjusting the pH of each solution to about pH 6.0 (using 1 M NaOH or 1 M HCl). For assays using Enterococcus faecalis (V583), individual growth media solutions for the assay were freshly prepared by adding 0.5 wt.% of a single polymer selected from Examples 5,7 and 13-14 to 1X TY media and then adjusting the pH of each solution to about pH 6.0 (using 1 M NaOH or 1 M HCl). All the growth media solutions were sterile filtered when possible using a 0.2 micrometer filter.

An aliquot of each growth media solution (200 microliters) was added to the well of a 96-well black, clear-bottom plate. Samples were prepared in triplicate (n=3). Reconstituted gelatin-fluorescein (20 microliters of 1 mg/mL) was then added to each well. The bacteria samples were resuspended in 5 mL of 10% TY media. A 3 microliter sample of either resuspended MPAO1-P2 Pseudomonas aeruginosa or Enterococcus faecalis (V583) was added to each well. Background control wells were also prepared that did not have bacteria added to the wells. Bacterial growth was measured at 600 nm (OD600) and collagenase activity was measured as fluorescent intensity at 485 nm excitation/528 nm emission. Collagenase activity (RFU) was normalized to bacterial growth (OD600) for each well. For MPAO1-P2 Pseudomonas aeruginosa, the time required to maximum fluorescence (i.e., time to inflection point) was measured. The test samples with added phosphonate-containing polymer had significantly longer times to reach the proteolytic activity inflection point than the control samples that had no added polymer (Tables 11, 12, and 13).

For Enterococcus faecalis, the collagenase activity was measured at the 12 hour time point. The test samples with added phosphonate-containing polymer had less collagenase production than the control samples that had no added polymer (Table 14). Compared to the control samples, the test samples with added phosphonate-containing polymer did not reduce bacterial growth.

TABLE 11

Time to the Proteolytic Activity Inflection Point (MPAO1-P2 P. aeruginosa)

| Polymer (0.5 wt.%) Added to Growth Media (10% TY Media) | Time to Proteolytic Activity Inflection Point (hours) |
|---|---|
| | Mean ± Standard Deviation (n = 3) |
| None (control) | 16.1 ± 1.6 |
| Example 1 | 21.3 ± 0.5 |
| Example 2 | 26.6 ± 4.6 |
| Example 3 | 27.7 ± 3.9 |
| Example 4 | 21.7 ± 0.2 |

TABLE 12

Time to the Proteolytic Activity Inflection Point (MPAO1-P2 P. aeruginosa)

| Polymer (0.05 wt.%) Added to Growth Media (10% TY Media) | Time to Proteolytic Activity Inflection Point (hours) |
|---|---|
| | Mean ± Standard Deviation (n = 3) |
| None (control) | 6.5 ± 0 |
| Example 15 | Did not reach inflection point by 30.2 hours |

TABLE 13

Time to the Proteolytic Activity Inflection Point (MPAO1-P2 P. aeruginosa)

| Polymer Added to Growth Media (10% TY Media) | Time to Proteolytic Activity Inflection Point (hours) |
|---|---|
| | Mean ± Standard Deviation (n = 3) |
| None (control) | 6.5 ± 0 |
| 0.1% wt.% Example 5 | 9.6 ± 0.3 |
| 0.1% wt.% Example 7 | Did not reach inflection point by 30.2 hours |
| 0.05 wt.% Example 5 + 0.05 wt.% Example 7 | Did not reach inflection point by 30.2 hours |
| 0.1 wt.% Example 5 + 0.1 wt.% Example 7 | Did not reach inflection point by 30.2 hours |

TABLE 14

Collagenase Production/Bacteria Growth at 12 hours (Enterococcus faecalis V583)

| Polymer (0.5 wt.%) Added to Growth Media (1X TY Media) | Collagenase Production (RFU/OD600) (12 hours) |
|---|---|
| | Mean ± Standard Deviation (n = 3) |
| None (control) | 2118 ± 523 |
| Example 5 | 1724 ± 38 |
| Example 7 | 905 ± 104 |
| Example 13 | 1591 ± 72 |
| Example 14 | 1171 ± 64 |

### Example 22: Biofilm Formation Assay Using Crystal Violet Staining

An MPAO1-P2 Pseudomonas aeruginosa colony from a TSA plate was grown overnight in 1X TY media (5 mL) with shaking at 37° C. Next, the bacteria were centrifuged at 3000 × g for 5 minutes and the supernatant was removed. The bacteria were washed once with water or 10% TY media.

Individual growth media solutions for the assay were freshly prepared by adding 0.5 wt.% of a single polymer selected from Examples 12-14 and Comparative Examples A-C to 10% TY media and then adjusting the pH of each solution to about pH 6.0 (using 1 M NaOH or 1 M HCl). Each growth media solution (200 microliters) was added to a separate well of a 96-well black, clear-bottom plate. Samples were prepared in triplicate (n=3). MPAO1-P2 bacteria were resuspended in 5 mL of 10% TY and 3 microliters of resuspended bacteria was added to each well. Background control wells were also prepared that did not have bacteria added to the wells. The 96-well plate was incubated at 37° C. with shaking for 20 hours using a plate reader to kinetically measure growth (OD600).

The solutions were aspirated from wells. Each well was washed two times with water (200 microliters per well) and then stained with 200 microliters of 0.1% aqueous crystal violet solution for 5 to 10 minutes. The crystal violet solution was then aspirated from each well and the wells were washed four times with water (200 microliters per wash per well). The remaining crystal violet stain in each well was solubilized with 200 microliters ethyl alcohol and then transferred to a well in a fresh 96-well plate. The absorbance of each well was measured at 550 nm and normalized to growth (background-subtracted OD600 measured at the 20 hour point of the kinetic growth measurements). The results are reported in Table 15. Compared to the control samples, the test samples with added phosphonate-containing polymer did not substantially reduce bacterial growth.

TABLE 15

Biofilm Results

| Polymer (0.5 wt.%) Added to Growth Media (10% TY Media) | Absorbance at 550 nm (from crystal violet stain) normalized to growth (OD600) at 20 hours |
|---|---|
| | Mean ± Standard Deviation (n = 3) |
| None (control) | 0.7 ± 0.1 |
| Example 12 | 0.24 ± 0.06 |
| Example 13 | 0.46 ± 0.05 |
| Example 14 | 0.9 ± 0.2 |
| Comparative Example A | 0.31 ± 0.07 |
| Comparative Example B | 0.4 ± 0.1 |
| Comparative Example C | 0.9 ± 0.2 |

What is claimed is:

1. A medical composition comprising:
a phosphonate-containing polymer having at least 0.8 mmoles phosphonate per gram of the phosphonate-containing polymer, wherein the phosphonate-containing polymer is a polymerized reaction product of a monomer composition comprising a first monomer of Formula (I) or a salt thereof 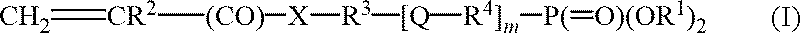
wherein
each R¹ is independently hydrogen, aryl, aralkyl, or alkaryl;
R² is hydrogen or methyl;
X is oxy or —NH—;
R³ is an alkylene or a heteroalkylene with one or more oxygen heteroatoms;
R⁴ is alkylene;
Q is —(CO)—O—, —NR⁵—(CO)—NR⁵—, —(CO)—NR⁵—, or —O—(CO)—NR⁵—;
R⁵ is hydrogen or alkyl,
m is equal to 0 or 1; and
wherein the medical composition is suitable for administration for preventing, mitigating, or treating a microbial infection.

2. (canceled)

3. The medical composition of claim 1, wherein the first monomer of Formula (I) is a monomer of Formula (I-C) or a salt thereof 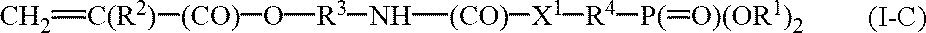
wherein X
¹ is oxy or —NR⁵—.

4. The medical composition of claim 1, wherein the first monomer of Formula (I) is a monomer of Formula (I-D) or a salt thereof 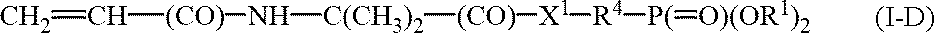
wherein X
¹ is oxy or —NR⁵—.

5. The medical composition of claim 1, wherein the monomer composition further comprises a second monomer that is hydrophilic and that comprises a) an ethylenically unsaturated group and b) a polar group that is an acidic group or salt thereof, a hydroxyl group, an ether, or a nitrogen-containing group.

6. The medical composition of claim 1, wherein the monomer composition further comprises a second monomer that comprises a) ethylenically unsaturated group and 2) a zwitterionic group.

7. The medical composition of claim 6, wherein the second monomer is of Formula (III) 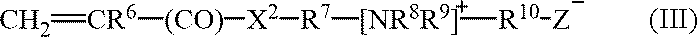
wherein
R⁶ is hydrogen or methyl;
X² is oxy or —NH—;
R⁷ is an alkylene or a heteroalkylene having an oxygen heteroatom; and
R⁸ and R⁹ are a) each independently an alkyl, aryl, aralkyl, or alkaryl, or b) R⁸ and R⁹ both combine with the nitrogen to which they are both attached to form a heterocyclic ring having 3 to 7 ring members; and
Z⁻ is carboxylate or sulfonate.

8. The medical composition of claim 5, wherein the phosphonate-containing polymer comprises greater than 25 mole percent of the first monomer.

9. The medical composition of claim 5, wherein the phosphonate-containing polymer comprises greater than 50 mole percent of the first monomer.

10. The medical composition of claim 1, wherein the medical composition is a spray, lotion, ointment, gel, solution, emulsion, dispersion, foam, coating, paste, powder, tablet, adhesive, or capsule.

11. A method of suppressing microbial virulence, the method comprising administrating and/or applying a medical composition of claim 1.

12. The method of claim 11, wherein the first monomer of Formula (I) is a monomer of Formula (I-C), Formula (I-D) or a salt thereof 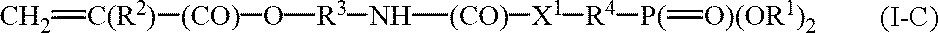 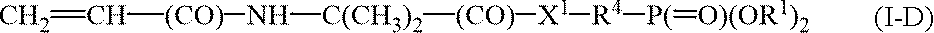
wherein X
¹ is oxy or —NR⁵—.

13. The method of claim 11, wherein administering the medical composition suppresses at least one type of virulence factor.

14. The method of claim 13, wherein the virulence factor is pyocyanin, pyoverdine, collagenase, or a biofilm.

15. The method of claim 14, wherein the biofilm is on a mammalian tissue or on a permanent or degradable implant.

16. The method of claim 11, wherein the log reduction of microbes is less than 1.

17. The method of claim 11, wherein administrating and/or applying the medical composition prevents, mitigates, or treats a microbial infection.

18. The method of claim 11, wherein administrating and/or applying the medical composition comprises applying the medical composition to skin, mucosa, tissue, a wound site, a surgical site, an implant, catheter, suture, or a bone.

19. The method of claim 11, wherein administrating and/or applying the medical composition reduces or inhibits virulence of at least one of gram negative Pseudomonas aeruginosa, gram positive Enterococcus faecalis, or gram positive Staphylococcus aureus.
